SEQUENCE LISTING (36 sequences)

SEQ ID NO: 1 - protein, 133 aa, synthetic construct [sequence omitted]

SEQ ID NO: 2 - protein, 133 aa, synthetic construct
APTSSSTKKTQLQLEHLLLDLQMILNGINNYKNPKLTRMLTEKFMMPKKATELKHLQCLEEELKPLEEVLNIAQSKNFHLRPRDLISNINVIVLELKGSETTFMCEYADETATIVEFLNRWITFGQSIISTLT

SEQ ID NO: 3 - protein, 133 aa, synthetic construct
APTSSSTKKTQLQLEHLLLDLQMILNGINNYKNPKLTRMLTWKFYMPKKATELKHLQCLEEELKPLEEVLNKAQSKNFHLRPRDLISNINVIVLELKGSETTFMCEYADETATIVEFLNRWITFCQSIISTLT

SEQ ID NO: 4 - protein, 133 aa, synthetic construct
APTSSSTKKTQLQLEHLLLDLQMILNGINNYKNPKLTRMLTVKFAMPKKATELKHLQCLEEELKPLEEVLNNAQSKNFHLRPRDLISNINVIVLELKGSETTFMCEYADETATIVEFLNRWITFGQSIISTLT

SEQ ID NO: 5 - protein, 133 aa, synthetic construct
APTSSSTKKTQLQLEHLLLDLQMILNGINNYKNPKLTRMLTYKFMMPKKATELKHLQCLEEELKPLEEVLNAAQSKNFHLRPRDLISNINVIVLELKGSETTFMCEYADETATIVEFLNRWITFYQSIISTLT

SEQ ID NO: 6 - protein, 133 aa, synthetic construct
APTSSSTKKTQLQLEHLLLDLQMILNGINNYKNPKLTRMLTMKFTMPKKATELKHLQCLEEELKPLEEVLNLAQSKNFHLRPRDLISNINVIVLELKGSETTFMCEYADETATIVEFLNRWITFYQSIISTLT

SEQ ID NO: 7 - protein, 133 aa, synthetic construct
APTSSSTKKTQLQLEHLLLDLQMILNGINNYKNPKLTRMLTMKFTMPKKATELKHLQCLEEELKPLEEVLNLAQSKNFHLRPRDLISNINVIVLELKGSETTFMCEYADETATIVEFLNRWITFRQSIISTLT

SEQ ID NO: 8 - protein, 133 aa, synthetic construct
APTSSSTKKTQLQLEHLLLDLQMILNGINNYKNPKLTRMLTEKFGMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 133 aa, synthetic construct
APTSSSTKKTQLQLEHLLLDLQMILNGINNYKNPKLTRMLTVKFSMPKKATELKHLQCLEEELKPLEEVLNNAQSKNFHLRPRDLISNINVIVLELKGSETTFMCEYADETATIVEFLNRWITFGQSIISTLT

SEQ ID NO: 10 - protein, 133 aa, synthetic construct
APTSSSTKKTQLQLEHLLLDLQMILNGINNYKNPKLTRMLTHKFDMPKKATELKHLQCLEEELKPLEEVLNIAQSKNFHLRPRDLISNINVIVLELKGSETTFMCEYADETATIVEFLNRWITFGQSIISTLT

SEQ ID NO: 11 - protein, 133 aa, synthetic construct
APTSSSTKKTQLQLEHLLLDLQMILNGINNYKNPKLTRMLTYKFMMPKKATELKHLQCLEEELKPLEEVLNAAQSKNFHLRPRDLISNINVIVLELKGSETTFMCEYADETATIVEFLNRWITFRQSIISTLT

SEQ ID NO: 12 - protein, 133 aa, synthetic construct
APTSSSTKKTQLQLEHLLLDLQMILNGINNYKNPKLTRMLTAKFLMPKKATELKHLQCLEEELKPLEEVLNNAQSKNFHLRPRDLISNINVIVLELKGSETTFMCEYADETATIVEFLNRWITFRQSIISTLT

SEQ ID NO: 13 - protein, 133 aa, synthetic construct
APTSSSTKKTQLQLEHLLLDLQMILNGINNYKNPKLTRMLTCKFNMPKKATELKHLQCLEEELKPLEEVLNVAQSKNFHLRPRDLISNINVIVLELKGSETTFMCEYADETATIVEFLNRWITFCQSIISTLT

SEQ ID NO: 14 - protein, 133 aa, synthetic construct
APTSSSTKKTQLQLEHLLLDLQMILNGINNYKNPKLTRMLTVKFPMPKKATELKHLQCLEEELKPLEEVLNPAQSKNFHLRPRDLISNINVIVLELKGSETTFMCEYADETATIVEFLNRWITFCQSIISTLT

SEQ ID NO: 15 - protein, 133 aa, synthetic construct
APTSSSTKKTQLQLEHLLLDLQMILNGINNYKNPKLTRMLTIKFVMPKKATELKHLQCLEEELKPLEEVLNKAQSKNFHLRPRDLISNINVIVLELKGSETTFMCEYADETATIVEFLNRWITFGQSIISTLT

SEQ ID NO: 16 - protein, 133 aa, synthetic construct
APTSSSTKKTQLQLEHLLLFLQMILNGINNYKTSDAKRNRNGYAGYIGPVSPYRRLSREKYLMQFIECDFNHFQSKNFHLRPRDLISYINVIVLELKGSETTFMCEYADETATIVEFLNRWITFGQSIISTLT

SEQ ID NO: 17 - protein, 133 aa, synthetic construct
APTSSSTKKTQLQLEHLLLDLQMILNGINNYKNPKLTRMLTQKFVMPKKATELKHLQCLEEELKPLEEVLNHAQSKNFHLRPRDLISNINVIVLELKGSETTFMCEYADETATIVEFLNRWITFCQSIISTLT

SEQ ID NO: 18 - protein, 133 aa, synthetic construct
APTSSSTKKTQLQLEHLLLDLQMILNGINNYKNPKLTRMLTFKFYMPKKATELKHLQCLEEELKPLEEVLNLAQSVNFHLRPRDLISNINVIVLELKGSETTFMCEYADETATIVEFLNRWITFCQSIISTLT

SEQ ID NO: 19 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFGQCIISTLT

SEQ ID NO: 20 - protein, 133 aa, synthetic construct
APTSSSTKKTQLQLEHLLLDLQMILNGINNYKNPMLTSMLPFGFTMPKKATELKHLQCLETELKPLEYVLNLAQSKNFHLRPRDLISNINVIVLELKGSETTFMCAYAWETATIVEFLNRWITFAQSIISTLT

SEQ ID NO: 21 - protein, 133 aa, synthetic construct
APTSSSTKKTQLQLEHLLLDLQMILNGINNYKNPKLTRMLTFKFYMPKKITELKHLQCLEEELKPLEEVLNLAQSKNFHLRPRDLISNINVIVLELKGSETTFMCEYADETATIVEFLNRWITFCQSIISTLT

SEQ ID NO: 22 - protein, 133 aa, synthetic construct
APTSSSTKKTQLQNEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 23 - protein, 133 aa, synthetic construct
APTSSSTKKTQLQLEHLLLDLQMILNGINNYKNPDLTSMLGFMFLMPKKATELKHLQCLEPELKPLELVLNLAQSKNFHLRPRDLISNINVIVLELKGSETTFMCMYARETATIVEFLNRWITFRQSIISTLT

SEQ ID NO: 24 - protein, 133 aa, synthetic construct
APTSSSTKKTQLQLEHLLLDLQMILNGINNYKNPKLTRMLTAKFDMPKKATELKHLQCLEEELKPLEEVLNDAQSKNFHLRPRDLISNINVIVLELKGSETTFMCEYADETATIVEFLNRWITFCQSIISTLT

SEQ ID NO: 25 - protein, 133 aa, synthetic construct
APTSSSTKKTQLQLEHLLLDLQMILNGINNYKNPKLTRMLTGKFCMPKKATELKHLQCLEEELKPLEEVLNLAQSKNFHLRPRDLISNINVIVLELKGSETTFMCEYADETATIVEFLNRWITFCQSIISTLT

SEQ ID NO: 26 - protein, 133 aa, synthetic construct
APTSSSTKKTQLQLEHLLLDLQMILNGINNYKNPKLTRMLTPKFIMPKKATELKHLQCLEEELKPLEEVLNCAQSKNFHLRPRDLISNINVIVLELKGSETTFMCEYADETATIVEFLNRWITFCQSIISTLT

SEQ ID NO: 27 - protein, 133 aa, synthetic construct
APTSSSTKKTQLQLEHLLLDLQMILNGINNYKNPKLTRMLTAKFMMPKKATELKHLQCLEEELKPLEEVLNNAQSKNFHLRPRDLISNINVIVLELKGSETTFMCEYADETATIVEFLNRWITFCQSIISTLT

SEQ ID NO: 28 - protein, 133 aa, synthetic construct
APTSSSTKKTQLQLEHLLLDLQMILNGINNYKNPKLTRMLTLKFTMPKKATELKHLQCLEEELKPLEEVLNGAQSKNFHLRPRDLISNINVIVLELKGSETTFMCEYADETATIVEFLNRWITFYQSIISTLT

SEQ ID NO: 29 - protein, 133 aa, synthetic construct; identical to SEQ ID NO: 2

SEQ ID NO: 30 - protein, 133 aa, synthetic construct
APTSSSTKKTQLQLEHLLLDLQMILNGINNYKNPKLTRMLTAKFAMPKKATELKHLQCLEEELKPLEEVLNNAQSKNFHLRPRDLISNINVIVLELKGSETTFMCEYADETATIVEFLNRWITFCQSIISTLT

SEQ ID NO: 31 - protein, 133 aa, synthetic construct
APTSSSTKKTQLQLEHLLLDLQMILNGINNYKNPKLTRMLTAKFDMPKKATELKHLQCLEEELKPLEEVLNNAQSKNFHLRPRDLISNINVIVLELKGSETTFMCEYADETATIVEFLNRWITFCQSIISTLT

SEQ ID NO: 32 - protein, 133 aa, synthetic construct
APTSSSTKKTQLQLEHLLLDLQMILNGINNYKNPKLTRMLTAKFGMPKKATELKHLQCLEEELKPLEEVLNNAQSKNFHLRPRDLISNINVIVLELKGSETTFMCEYADETATIVEFLNRWITFCQSIISTLT

SEQ ID NO: 33 - protein, 133 aa, synthetic construct
APTSSSTKKTQLQLEHLLLDLQMILNGINNYKNPKLTRMLTAKFQMPKKATELKHLQCLEEELKPLEEVLNNAQSKNFHLRPRDLISNINVIVLELKGSETTFMCEYADETATIVEFLNRWITFCQSIISTLT

SEQ ID NO: 34 - protein, 133 aa, synthetic construct
APTSSSTKKTQLQLEHLLLDLQMILNGINNYKNPKLTRMLTAKFRMPKKATELKHLQCLEEELKPLEEVLNNAQSKNFHLRPRDLISNINVIVLELKGSETTFMCEYADETATIVEFLNRWITFCQSIISTLT

SEQ ID NO: 35 - protein, 133 aa, synthetic construct
APTSSSTKKTQLQLEHLLLDLQMILNGINNYKNPKLTRMLTAKFSMPKKATELKHLQCLEEELKPLEEVLNNAQSKNFHLRPRDLISNINVIVLELKGSETTFMCEYADETATIVEFLNRWITFCQSIISTLT

SEQ ID NO: 36 - protein, 133 aa, synthetic construct
APTSSSTKKTQLQLEHLLLDLQMILNGINNYKNPKLTRMLTAKFTMPKKATELKHLQCLEEELKPLEEVLNNAQSKNFHLRPRDLISNINVIVLELKGSETTFMCEYADETATIVEFLNRWITFCQSIISTLT

(19) United States
(12) Patent Application Publication
(10) Pub. No.: US 2026/0028383 A1
(43) Pub. Date: Jan. 29, 2026

(54) INTERLEUKIN-2 VARIANTS AND THEIR USES IN TREATING CANCERS

(71) Applicant: KINE BIOTECH CO., LTD., Taipei City (TW)

(72) Inventors: Keng-Li LAN, Taipei City (TW); Cheng-Liang TSAI, New Taipei City (TW); Yen-Hua HUANG, Taipei City (TW)

(21) Appl. No.: 18/998,124

(22) PCT Filed: Aug. 16, 2023
(86) PCT No.: PCT/CN2023/113400

§ 371 Date: Jan. 24, 2025

Related U.S. Application Data
(60) Provisional application No. 63/398,290, filed on Aug. 16, 2022.

(51) Int. Cl. C07K 14/55; A61K 38/00; A61P 35/00

(52) U.S. Cl. CPC C07K 14/55; A61P 35/00; A61K 38/00

(57) ABSTRACT

Disclosed herein is generally related to an isolated interleukin-2 (IL-2) variant that exhibits reduced affinity to the α-subunit of IL-2 receptor (IL-2R), for use as immunotherapeutic agents. In addition, the present disclosure relates to a pharmaceutical composition comprising the isolated IL-2 variant, and uses thereof in treating a cancer in a subject.

### CROSS-REFERENCE OF RELATED APPLICATION

This application claims priority to U.S. Application No. 63/398,290, filed on Aug. 16, 2022. The content of which application is incorporated herein by reference in its entirety.

### SEQUENCE LISTING XML

The present application is being filed along with a Sequence Listing XML in electronic format. The Sequence Listing XML is provided as an XML file entitled “P4261-PCT_SeqList_20230804_filed.xml,” created Aug. 4, 2023, which is 36 Kb in size. The information in the electronic format of the Sequence Listing XML is incorporated herein by reference in its entirety.

### BACKGROUND OF THE INVENTION

### 1. Field of the Invention

The present disclosure in general relates to the field of cancer treatment. More particularly, the present disclosure relates to interleukin-2 (IL-2) variants and their uses for treating cancers.

### 2. Description of Related Art

Cancer is a group of diseases involving an uncontrolled division of abnormal cells in a part of the body with the potential to invade or spread to other parts of the body. For a long time, cancer is a severe health threat to all human beings and is a heavy burden for health care system of a society. According to statistics provided by the World Health Organization, in 2020 alone, there are a total of 19,292,789 new cases and 9,958,133 deaths for all cancers in both sexes and in all ages, leading to a significant loss of human life and economy.

As such, scientists around the world are committed to developing effective cancer treatments to improve the efficacy of treating cancers, and up to date, there are current therapeutics including surgery, chemotherapy, radiation therapy, hormonal therapy, thermal therapy, palliative care, and the like being employed in treating cancers. However, such conventional approach is often accompanied with drastic side effects such as injury or cytotoxicity to normal tissues. In light of the issue as set forth above, treatments with fewer side-effects have been developed accordingly in recent years, some of which are under research or in preclinical stage. Among these treatments, immunotherapy using biological macromolecule (e.g., polypeptides, nucleic acids, carbohydrates, and etc.) is viewed as the most promising treatment, as it eliminates tumors via modulating the function of immune system of the host, leading to minimizing possible side-effects imposed on the patient.

IL-2 is a potential immunotherapeutic agent in treating cancers. Wild-type IL-2 (also named as proleukin, a T cell growth factor (TCGF)), a 15.5 kDa globular glycoprotein involving in lymphocyte generation, survival and homeostasis. IL-2 is synthesized mainly by activated T-cells, in particular CD4⁺ helper T cells. It stimulates the proliferation and differentiation of T cells, induces the generation of cytotoxic T lymphocytes (CTLs), the differentiation of peripheral blood lymphocytes to cytotoxic cells and lymphokine-activated killer (LAK) cells, promotes cytokine and cytolytic molecule expression by T cells, facilitates the proliferation and differentiation of B-cells and the synthesis of immunoglobulin by B-cells, and stimulates the generation, proliferation and activation of natural killer (NK) cells. With the ability of IL-2 to expand lymphocyte populations in vivo and to increase the effector functions of these cells that confers anti-tumor effects, IL-2 has been considered as an attractive treatment option for treating cancers.

However, IL-2 has a dual function in the immune response in that it not only mediates expansion and activity of effector cells, but also is crucially involved in maintaining peripheral immune tolerance. IL-2 mediates its action by binding to IL-2 receptor (IL-2R), which consist of the α-subunit (CD25), β-subunit (CD122), and γ_{c}-subunit (γ_{c}, CD132) subunits in dimeric form (containing the β/γ_{c}-subunits) or in trimeric form (containing the α/β/γ_{c}-subunits). Among the foregoing subunits, the α-subunit confers high-affinity binding to its receptor, whereas the β-subunit and the γ_{c}-subunit are crucial for signal transduction. For the immune tolerance, IL-2 is involved in part by binding to the trimeric (i.e., the α/β/γ_{c}-subunits) IL-2R that are expressed by regulatory T (T_{reg}) cells, which, after binding, activates the activities of the T_{reg} cells that in turn triggers the immunosuppression of the surroundings, leading to compromising the host's anti-tumor immunity. Accordingly, wild-type IL-2 is not optimal for inhibiting tumor growth, and optimization of IL-2, by screening a series of IL-2 variants in order to seek the ones with reduced affinity to the α-subunit of IL-2R, while the affinity to the β-subunit and the γ_{c}-subunit of IL-2R remained not affected, may help to reduce immunosuppression caused by T_{reg} cells, thereby enhancing the overall anti-tumor activities of IL-2.

In view of the foregoing, there exists in the related art a need for seeking optimal IL-2 variants to overcome the problems associated with IL-2 immunotherapy, by trimming the unwanted immune tolerance activities of IL-2 protein so as to enhance its therapeutic usefulness.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to an isolated IL-2 variant that exhibits reduced affinity to the α-subunit of IL-2R, while the affinity to the β-subunit and the γ_{c}-subunit of IL-2R remained unaffected, having an amino acid sequence at least 85% identical to SEQ ID NO: 1, wherein the isolated IL-2 variant comprises an amino acid substitution at a position corresponding to the positions 42, 45, 50, 72, or 125 in SEQ ID NO: 1, in which:
the amino acid substitution at the position corresponding to the position 42 in SEQ ID NO: 1 is a substitution of phenylalanine (F) with alanine (A), cysteine (C), glutamic acid (E), histidine (H), valine (V), or tryptophan (W);
the amino acid substitution at the position corresponding to the position 45 in SEQ ID NO: 1 is a substitution of tyrosine (Y) with alanine (A), aspartic acid (D), glycine (G), methionine (M), asparagine (N), glutamine (Q), arginine (R), serine(S), or threonine (T);
the amino acid substitution at the position corresponding to the position 50 in SEQ ID NO: 1 is a substitution of alanine (A) with isoleucine (I);
the amino acid substitution at the position corresponding to the position 72 in SEQ ID NO: 1 is a substitution of leucine (L) with aspartic acid (D), isoleucine (I), lysine (K), asparagine (N), or valine (V); and
the amino acid substitution at the position corresponding to the position 125 in SEQ ID NO: 1 is a substitution of cysteine (C) with glycine (G).

According to some embodiments of the present disclosure, the isolated IL-2 variant comprises an amino acid sequence at least 85% identical to SEQ ID NO: 1 with the amino acid substitution of phenylalanine (F) with tryptophan (W) at the position corresponding to the position 42 in SEQ ID NO: 1, and the amino acid substitution of leucine (L) with lysine (K) at the position corresponding to the position 72 in SEQ ID NO: 1.

According to other embodiments of the present disclosure, the isolated IL-2 variant comprises an amino acid sequence at least 85% identical to SEQ ID NO: 1 with the amino acid substitution of phenylalanine (F) with valine (V) at the position corresponding to the position 42 in SEQ ID NO: 1, the amino acid substitution of tyrosine (Y) with alanine (A) at the position corresponding to the position 45 in SEQ ID NO: 1, the amino acid substitution of leucine (L) with asparagine (N) at the position corresponding to the position 72 in SEQ ID NO: 1, and the amino acid substitution of cysteine (C) with glycine (G) at the position corresponding to the position 125 in SEQ ID NO: 1.

According to still other embodiments of the present disclosure, the isolated IL-2 variant comprises an amino acid sequence at least 85% identical to SEQ ID NO: 1 with the amino acid substitution of phenylalanine (F) with glutamic acid (E) at the position corresponding to the position 42 in SEQ ID NO: 1, and the amino acid substitution of tyrosine (Y) with glycine (G) at the position corresponding to the position 45 in SEQ ID NO: 1.

Optionally, the isolated IL-2 variant of the present disclosure comprises an amino acid sequence at least 85% identical to SEQ ID NO: 1 with the amino acid substitution of phenylalanine (F) with valine (V) at the position corresponding to the position 42 in SEQ ID NO: 1, the amino acid substitution of tyrosine (Y) with serine(S) at the position corresponding to the position 45 in SEQ ID NO: 1, the amino acid substitution of leucine (L) with asparagine (N) at the position corresponding to the position 72 in SEQ ID NO: 1, and the amino acid substitution of cysteine (C) with glycine (G) at the position corresponding to the position 125 in SEQ ID NO: 1.

Alternatively or in addition, the isolated IL-2 variant of the present disclosure comprises an amino acid sequence at least 85% identical to SEQ ID NO: 1 with the amino acid substitution of phenylalanine (F) with histidine (H) at the position corresponding to the position 42 in SEQ ID NO: 1, the amino acid substitution of tyrosine (Y) with aspartic acid (D) at the position corresponding to the position 45 in SEQ ID NO: 1, the amino acid substitution of leucine (L) with isoleucine (I) at the position corresponding to the position 72 in SEQ ID NO: 1, and the amino acid substitution of cysteine (C) with glycine (G) at the position corresponding to the position 125 in SEQ ID NO: 1.

In some further embodiments of the present disclosure, the isolated IL-2 variant comprises an amino acid sequence at least 85% identical to SEQ ID NO: 1 with the amino acid substitution of phenylalanine (F) with cysteine (C) at the position corresponding to the position 42 in SEQ ID NO: 1, the amino acid substitution of tyrosine (Y) with asparagine (N) at the position corresponding to the position 45 in SEQ ID NO: 1, and the amino acid substitution of leucine (L) with valine (V) at the position corresponding to the position 72 in SEQ ID NO: 1.

Alternatively, in a certain embodiment of the present disclosure, the isolated IL-2 variant comprises an amino acid sequence at least 85% identical to SEQ ID NO: 1 with the amino acid substitution of alanine (A) with isoleucine (I) at the position corresponding to the position 50 in SEQ ID NO: 1.

According to some particular embodiments of the present disclosure, the isolated IL-2 variant comprises an amino acid sequence at least 85% identical to SEQ ID NO: 1 with the amino acid substitution of phenylalanine (F) with alanine (A) at the position corresponding to the position 42 in SEQ ID NO: 1, the amino acid substitution of tyrosine (Y) with aspartic acid (D) at the position corresponding to the position 45 in SEQ ID NO: 1, and the amino acid substitution of leucine (L) with aspartic acid (D) at the position corresponding to the position 72 in SEQ ID NO: 1.

Yet in some further embodiments of the present disclosure, the isolated IL-2 variant comprises an amino acid sequence at least 85% identical to SEQ ID NO: 1 with the amino acid substitution of phenylalanine (F) with alanine (A) at the position corresponding to the position 42 in SEQ ID NO: 1, the amino acid substitution of tyrosine (Y) with methionine (M) at the position corresponding to the position 45 in SEQ ID NO: 1, and the amino acid substitution of leucine (L) with asparagine (N) at the position corresponding to the position 72 in SEQ ID NO: 1.

Still yet in some further embodiments of the present disclosure, the isolated IL-2 variant comprises an amino acid sequence at least 85% identical to SEQ ID NO: 1 with the amino acid substitution of phenylalanine (F) with alanine (A) at the position corresponding to the position 42 in SEQ ID NO: 1, the amino acid substitution of tyrosine (Y) with arginine (R) at the position corresponding to the position 45 in SEQ ID NO: 1, and the amino acid substitution of leucine (L) with asparagine (N) at the position corresponding to the position 72 in SEQ ID NO: 1.

According to some prefer embodiments of the present disclosure, the isolated IL-2 variant comprises an amino acid sequence at least 90% identical to SEQ ID NO: 1; more preferably, the isolated IL-2 variant comprises an amino acid sequence at least 95% identical to SEQ ID NO: 1; even more preferably, the isolated IL-2 variant comprises the amino acid sequence of SEQ ID NO: 1.

Accordingly, encompassed in the present disclosure is a pharmaceutical composition comprising the foregoing isolated IL-2 variant, and a pharmaceutically acceptable carrier.

Another aspect of the present disclosure pertains to a method for treating a cancer in a subject with the aid of the present isolated IL-2 variant or the present pharmaceutical composition comprising the same, which comprises the step of administering to the subject an effective amount of the present isolated IL-2 variant or the present the pharmaceutical composition.

According to some preferred embodiments of the present disclosure, the present isolated IL-2 variant or the present pharmaceutical composition is administered to the subject in the amount of about 1-500 μg/kg body weight.

The cancer that is treatable by the present isolated IL-2 variant, the present pharmaceutical composition, or the present method may be bladder cancer, biliary cancer, bone cancer, brain tumor, breast cancer, cervical cancer, colorectal cancer, esophageal cancer, epidermal carcinoma, gastric cancer, gastrointestinal stromal tumor (GIST), glioma, hematopoietic tumors of lymphoid lineage, hepatic cancer, Kaposi's sarcoma, leukemia, lung cancer, lymphoma, intestinal cancer, melanoma, myeloid leukemia, pancreatic cancer, prostate cancer, retinoblastoma, ovary cancer, renal cell carcinoma, spleen cancer, squamous cell carcinoma, thyroid cancer, or thyroid follicular cancer.

According to some advanced embodiments of the present disclosure, the present method further comprises the step of subjecting the subject in need to a surgery, a radiotherapy, a chemotherapy, an immunotherapy, a hormone therapy, or a combination therapy thereof, prior to, concurrently with, or subsequent to the administration of the present isolated IL-2 variant or the present pharmaceutical composition.

In some preferred embodiments of the present disclosure, the immunotherapy is used in combination with the present isolated IL-2 variant or the present pharmaceutical composition in the present method, in which said immunotherapy comprises administering to the subject an effective amount of C-C motif chemokine ligand 3 (CCL3), C-C motif chemokine ligand 26 (CCL26), C-X-C motif chemokine ligand 7 (CXCL7); granulocyte colony-stimulating factor (G-CSF), granulocyte macrophage colony-stimulating factor (GM-CSF), interferon-α (IFN-α), interferon-β (IFN-β), interferon-γ (IFN-γ), tumor necrosis factor-α (TNF-α); interleukin-4 (IL-4), interleukin-5 (IL-5), interleukin-7 (IL-7), interleukin-10 (IL-10), interleukin-12 (IL-12), interleukin-13 (IL-13), interleukin-15 (IL-15); apremilast, imiquimod, lenalidomide, pomalidomide, sipuleucel-T, thalidomide; anti-CTLA-4 antibody, anti-PD-1 antibody, anti-PD-L1 antibody; anti-CD2 antibody, anti-CD3 antibody, anti-CD4 antibody, anti-CD11a antibody, anti-CD20 antibody, anti-CD25 antibody, anti-CD52 antibody, anti-EGFR antibody, anti-HER2 antibody, anti-PCDP1 antibody, anti-SLAMF7 antibody, or anti-Trop-2 antibody. According to more preferred embodiments of the present disclosure, the immunotherapy used in combination with the present isolated IL-2 variant or the present pharmaceutical composition in the present method comprises administering to the subject an effective amount of IL-15.

The subject treatable by the present isolated IL-2 variant, the present pharmaceutical composition, or the present method is a mammal, for example, a human, a mouse, a rat, a guinea pig, a hamster, a monkey, a swine, a dog, a cat, a horse, a sheep, a goat, a cow, and a rabbit. Preferably, the subject is a human.

Many of the attendant features and advantages of the present disclosure will becomes better understood with reference to the following detailed description considered in connection with the accompanying drawings.

### BRIEF DESCRIPTION OF THE DRAWINGS

These and other features, aspects and advantages of the present invention will become better understood with reference to the following description, appended claims and the accompanying drawings, where:

FIGS. 1A-1E are the characterization results of the present IL-2 variants prepared in accordance with the embodiments of the present disclosure. FIG. 1A is the western blotting results depicting the expression of the indicated IL-2 variants. FIGS. 1B-1E are the flow cytometry results depicting the level of phosphorylated STAT-5 (pSTAT-5) in CTLL-2 cells (expressing the α/β/γ_{c}-subunits of IL-2R; FIG. 1B) or in HH ells (expressing the β/γ_{c}-subunits of IL-2R; FIGS. 1C-1E) induced by the indicated IL-2 variants (30 nM each in FIGS. 1B, and 1D-1E; 4 nM in FIG. 1C). MFI, mean fluorescence intensity; % of pSTAT-5⁺ cells, showing the percentage of pSTAT-5-positive cells.

FIGS. 2A-2F are the flow cytometry results depicting the level of pSTAT-5 in CTLL-2 cells (FIGS. 2A, 2C, and 2E) or in HH ells (FIGS. 2B, 2D, and 2F) induced by a series concentration (0-30 nM) of the indicated IL-2 variants. IL-2v (AAG), a mutant IL-2 (AAG: with F42A, Y45A, and L72G substitutions of IL-2).

FIGS. 3A-3D are the relative organ weight (FIG. 3A, lung; FIG. 3B, liver; FIG. 3C, spleen; relative to the body weight) and the body weight (FIG. 3D) of the mice treated with the indicated treatments. **, p<0.01; ***, p<0.005; ****, p<0.0001.

FIGS. 4A-4C are the flow cytometry results depicting active CD8⁺ T cells (FIG. 4A), active NK cells (FIG. 4B), and T_{reg} cells (FIG. 4C) in the spleen of the mice treated with the indicated treatments. **, p<0.01; ***, p<0.005; ****, p<0.0001.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a,” “an,” and “the” include the plural reference unless the context clearly dictates otherwise. Also, as used herein and in the claims, the terms “at least one” and “one or more” have the same meaning and include one, two, three, or more. The practice of the present invention will employ, unless otherwise indicated, conventional techniques of molecular biology, tumor biology, microbiology, and immunology, which are within the skill of the art. Such techniques are explained fully in the literature.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “interleukin-2” or “IL-2” as used herein, refers to any native IL-2 polypeptide from any vertebrate source, including mammals such as primates (e.g. humans) and rodents (e.g., mice and rats), unless otherwise indicated. The term encompasses unprocessed IL-2 as well as any form of IL-2 that results from processing in the cell. The term also encompasses naturally occurring variants of IL-2, e.g. splice variants or allelic variants. The amino acid sequence of an exemplary human IL-2 is shown in SEQ ID NO: 1. Unprocessed human IL-2 additionally comprises an N-terminal 20 amino acid signal peptide, which is absent in the mature IL-2 molecule.

The term “IL-2 variant” as used herein is intended to encompass any mutant forms of various forms of the IL-2 polypeptide including full-length IL-2, truncated forms of IL-2 and forms where IL-2 is linked to another molecule such as by fusion or chemical conjugation. “Full-length” when used in reference to IL-2 is intended to mean the mature, natural length IL-2 molecule. For example, full-length human IL-2 refers to a molecule that has 133 amino acids (see e.g. SEQ ID NO: 1). The various forms of IL-2 variants are characterized in having a at least one amino acid mutation affecting the interaction of IL-2 with the α-subunit of the IL-2R, CD25. This mutation may involve substitution, deletion, truncation, or modification of the wild-type amino acid residue normally located at that position. Mutants obtained by amino acid substitution are preferred. Unless otherwise indicated, an IL-2 variant may be referred to herein as an IL-2 mutant, an IL-2 mutant peptide sequence, an IL-2 mutant polypeptide, IL-2 mutant protein, or IL-2 mutant analog. Designation of various forms of IL-2 is herein made with respect to the sequence shown in SEQ ID NO: 1. Various designations may be used herein to indicate the same mutation. For example a mutation from phenylalanine at position 42 to alanine can be indicated as 42A, A42, F42A, or Phe42Ala.

As used herein, a “wild-type” form of IL-2 is a form of IL-2 that is otherwise the same as the IL-2 variant except that the wild-type form has a wild-type amino acid at each amino acid position of the IL-2 variant. For example, if the IL-2 variant is the full-length IL-2 (i.e. IL-2 not fused or conjugated to any other molecule), the wild-type form of this mutant is full-length native IL-2. If the IL-2 variant is a fusion between IL-2 and another polypeptide encoded downstream of IL-2 (e.g. an antibody chain), the wild-type form of this IL-2 variant is IL-2 with a wild-type amino acid sequence fused to the same downstream polypeptide. Furthermore, if the IL-2 variant is a truncated form of IL-2 (the mutated or modified sequence within the non-truncated portion of IL-2), then the wild-type form of this IL-2 variant is a similarly truncated IL-2 that has a wild-type sequence. For the purpose of comparing IL-2 receptor binding affinity or biological activity of various forms of IL-2 variants to the corresponding wild-type form of IL-2, the term wild-type encompasses forms of IL-2 comprising one or more amino acid mutation that does not affect IL-2 receptor binding compared to the naturally occurring, native IL-2, such as e.g. a substitution of cysteine at a position corresponding to residue 125 of human IL-2 to alanine.

The term “high-affinity IL-2R” as used herein refers to the heterotrimeric form of the IL-2R, consisting of the receptor α-subunit (also known as CD25 or p55), the receptor β-subunit (also known as CD122 or p70), and the receptor γ_{c}-subunit (also known as common cytokine receptor γ_{c}-subunit, γ_{c}, or CD132). The term “intermediate-affinity IL-2R” by contrast refers to the IL-2R including only the β-subunit and the γ_{c}-subunit, without the α-subunit.

As used herein, the term “affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., receptor and a ligand). The affinity of a molecule-X for its partner Y can generally be represented by the dissociation constant (K_{D}), which is the ratio of dissociation and association rate constants (K_{off} and Kₒₙ, respectively). Thus, equivalent affinities may comprise different rate constants, as long as the ratio of the rate constants remains the same. Affinity may be measured by any well-established methods known in the art, such as analysis by surface plasmon resonance (SPR), using standard instrumentation such as a BIAcore instrument, in which each subunit of the IL-2R may be obtained by recombinant expression. Alternatively, binding affinity of IL-2 variants for different forms of the IL-2R may be evaluated using cell lines known to express one or the other such form of the IL-2R.

As used herein, the term “effector cells” refers to a population of lymphocytes that mediate the cytotoxic effects of IL-2. Effector cells include effector T cells such as CD8⁺ cytotoxic T cells, NK cells, LAK cells and macrophages/monocytes.

As used herein, the term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended to refer to a polypeptide that is not in its natural milieu. No particular level of purification is required. For example, an isolated polypeptide may be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

As discussed herein, minor variations in the amino acid sequences of the present IL-2 variant are contemplated as being encompassed by the presently disclosed and claimed inventive concept(s), providing that the variations in the amino acid sequence of the present IL-2 variant occur at positions other than that corresponding to the positions 42, 45, 50, 72, or 125 in SEQ ID NO: 1 (i.e., the positions 1-41, 43-44, 46-49, 51-71, 73-124, or 126-133 in SEQ ID NO: 1), and maintain at least 85% sequence identity to SEQ ID NO: 1, such as 85%, 86%, 87%, 88%, 89%, 90%, 91%, 92%, 93%, 94%, 95%, 96%, 97%, 98%, 99%, and 100% sequence identity to SEQ ID NO: 1, without affecting the binding affinity of the present IL-2 variant to the α-subunit of the IL-2R.

“Percentage (%) sequence identity” is defined as the percentage of amino acid residues in a candidate sequence that are identical with the amino acid residues in the specific peptide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{X}{Y} \times 100$$

where X is the number of amino acid residues scored as identical matches by the sequence alignment program BLAST in that program's alignment of A and B, and where Y is the total number of amino acid residues in A or B, whichever is shorter.

The term “pharmaceutical composition” refers to a preparation which is in such form as to permit the biological activity of an active ingredient contained therein to be effective, and which contains no additional components which are unacceptably toxic to a subject to which the composition would be administered.

The phrase “pharmaceutically acceptable carrier” as used herein means a pharmaceutically acceptable material, composition or vehicle, such as a liquid or solid filler, diluent, carrier, solvent or encapsulating material, involved in carrying or transporting the subject agents from one organ, or portion of the body, to another organ, or portion of the body. Each carrier must be “acceptable” in the sense of being compatible with the other ingredients of the formulation. The pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually, the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, the pharmaceutical composition of the invention is formulated into formulations suitable for the intended route of administration.

The terms “treatment” and “treating” as used herein may refer to a curative or palliative measure. In particular, the term “treating” as used herein refers to the application or administration of the present IL-2 variant or a pharmaceutical composition comprising the same to a subject, who has a cancer, a symptom associated with a cancer, a disease or disorder secondary to a cancer, with the purpose to partially or completely alleviate, ameliorate, relieve, delay onset of, inhibit progression of, reduce severity of, and/or reduce incidence of one or more symptoms or features of a cancer.

The term “administered,” “administering” or “administration” are used interchangeably herein to refer means either directly administering the present IL-2 variant, the pharmaceutical composition comprising the present IL-2 variant, and/or the method of the present invention.

The term “an effective amount” as used herein refers to an amount effective, at dosages, and for periods of time necessary, to achieve the desired therapeutically desired result with respect to the treatment of cancers. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/kg). Alternatively, the effective amount can be expressed in the concentration of the active component (e.g., the present IL-2 variant), such as molar concentration, mass concentration, volume concentration, molality, mole fraction, mass fraction and mixing ratio. Persons having ordinary skills could calculate the human equivalent dose (HED) for the medicament (such as the present antibody) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “subject” or “patient” refers to an animal including the human species that is treatable with the IL-2 variant, the pharmaceutical composition and/or the method of the present disclosure. The term “subject” or “patient” intended to refer to both the male and female gender unless one gender is specifically indicated. Accordingly, the term “subject” or “patient” comprises any mammal which may benefit from treatment of cancer. In an exemplary embodiment, the patient is a human.

### II. Description of the Invention

The present disclosure is based, at least in part, on the discovery that the present IL-2 variant possesses reduced or abolished affinity to the α-subunit of the IL-2R, making the present IL-2 variant having improved properties for immunotherapy. As provided above, different forms of the IL-2R are made of different subunits and exhibit different affinities for IL-2. The intermediate-affinity IL-2R having the receptor β/γ_{c}-subunits is expressed on resting effector cells like NK and T cells, and is sufficient for IL-2 signaling, after its engagement by IL-2, to induce proliferation and activation of these effector cells. By contrast, the high-affinity IL-2R further comprising the receptor α-subunit is mainly expressed on T_{reg} cells as well as on activated effector cells, where its binding with IL-2 is capable of promoting T_{reg} cell-mediated immunosuppression or activation-induced cell death (AICD), respectively. Accordingly, lowering the affinity of IL-2 to the α-subunit of the IL-2R may reduce IL-2 induced downregulation of function of effector cells by T_{reg} cells, as well as curb development of tumor tolerance by the process of AlCD. Thus, the main purpose of the present disclosure is providing the IL-2 variant with lower affinity to the α-subunit of the IL-2R so as to confer the desired characteristics of IL-2 for immunotherapy.

### 1. The IL-2 Variant

Accordingly, the first aspect of the present disclosure pertains to an isolated IL-2 variant comprising at least one amino acid mutation that leads to reducing or abolishing affinity of the IL-2 variant to the α-subunit of the IL-2R while preserves affinity of the IL-2 variant to the intermediate-affinity IL-2R, as compared to a wild-type IL-2. The isolated IL-2 variant with decreased affinity to the α-subunit of the IL-2R may have an amino acid sequence at least 85% identical to SEQ ID NO: 1, where the at least one amino acid substitution thereon occurs at a position corresponding to the positions 42, 45, 50, 72, or 125 in SEQ ID NO: 1, in which:
the amino acid substitution at the position corresponding to the position 42 in SEQ ID NO: 1 is a substitution of phenylalanine (F) with alanine (A), cysteine (C), glutamic acid (E), histidine (H), valine (V), or tryptophan (W);
the amino acid substitution at the position corresponding to the position 45 in SEQ ID NO: 1 is a substitution of tyrosine (Y) with alanine (A), aspartic acid (D), glycine (G), methionine (M), asparagine (N), glutamine (Q), arginine (R), serine(S), or threonine (T);
the amino acid substitution at the position corresponding to the position 50 in SEQ ID NO: 1 is a substitution of alanine (A) with isoleucine (I);
the amino acid substitution at the position corresponding to the position 72 in SEQ ID NO: 1 is a substitution of leucine (L) with aspartic acid (D), isoleucine (I), lysine (K), asparagine (N), or valine (V); and
the amino acid substitution at the position corresponding to the position 125 in SEQ ID NO: 1 is a substitution of cysteine (C) with glycine (G);
   these mutants exhibit substantially similar binding affinity to the intermediate-affinity IL-2R, and have substantially reduced affinity to the α-subunit of the IL-2R or the high-affinity IL-2R as compared to a wild-type form of the IL-2.

In accordance with the embodiments of the present disclosure, the present IL-2 variant may have: an amino acid sequence at least 85% identical to SEQ ID NO: 1, such as 85%, 86%, 87%, 88%, 89%, 90%, 91%, 92%, 93%, 94%, 95%, 96%, 97%, 98%, 99%, and 100% sequence identity to SEQ ID NO: 1; preferably, an amino acid sequence at least 90% identical to SEQ ID NO: 1; more preferably, an amino acid sequence at least 95% identical to SEQ ID NO: 1; even more preferably, the amino acid sequence of SEQ ID NO: 1; with the at least one amino acid substitutions occurring at a relative position corresponding to the positions 42, 45, 50, 72, or 125 in SEQ ID NO: 1, and such amino acid substitutions are selected from the group consisting of F42A, F42C, F42E, F42H, F42V, and F42W; Y45A, Y45D, Y45G, Y45M, Y45N, and Y45S; A501; L72D, L72I, L72K, L72N, and L72V; and C125G.

According to some embodiments of the present disclosure, the present IL-2 variant term as IL-2_#2 may have: an amino acid sequence at least 85% identical to SEQ ID NO: 1; preferably, an amino acid sequence at least 90% identical to SEQ ID NO: 1; more preferably, an amino acid sequence at least 95% identical to SEQ ID NO: 1; even more preferably, the amino acid sequence of SEQ ID NO: 1; with the following amino acid substitutions: the amino acid substitution of phenylalanine (F) with tryptophan (W) at the position corresponding to the position 42 in SEQ ID NO: 1, and the amino acid substitution of leucine (L) with lysine (K) at the position corresponding to the position 72 in SEQ ID NO: 1. In one working embodiment, the present IL-2 variant IL-2_#2 has the amino acid sequence of SEQ ID NO: 3.

According to other embodiments of the present disclosure, the present IL-2 variant term as IL-2_#3 may have: an amino acid sequence at least 85% identical to SEQ ID NO: 1; preferably, an amino acid sequence at least 90% identical to SEQ ID NO: 1; more preferably, an amino acid sequence at least 95% identical to SEQ ID NO: 1; even more preferably, the amino acid sequence of SEQ ID NO: 1; with the following amino acid substitutions: the amino acid substitution of phenylalanine (F) with valine (V) at the position corresponding to the position 42 in SEQ ID NO: 1, the amino acid substitution of tyrosine (Y) with alanine (A) at the position corresponding to the position 45 in SEQ ID NO: 1, the amino acid substitution of leucine (L) with asparagine (N) at the position corresponding to the position 72 in SEQ ID NO: 1, and the amino acid substitution of cysteine (C) with glycine (G) at the position corresponding to the position 125 in SEQ ID NO: 1. In one working embodiment, the present IL-2 variant IL-2_#3 has the amino acid sequence of SEQ ID NO: 4.

According to still other embodiments of the present disclosure, the present IL-2 variant term as IL-2_#7 may have: an amino acid sequence at least 85% identical to SEQ ID NO: 1; preferably, an amino acid sequence at least 90% identical to SEQ ID NO: 1; more preferably, an amino acid sequence at least 95% identical to SEQ ID NO: 1; even more preferably, the amino acid sequence of SEQ ID NO: 1; with the following amino acid substitutions: the amino acid substitution of phenylalanine (F) with glutamic acid (E) at the position corresponding to the position 42 in SEQ ID NO: 1, and the amino acid substitution of tyrosine (Y) with glycine (G) at the position corresponding to the position 45 in SEQ ID NO: 1. In one working embodiment, the present IL-2 variant IL-2_#7 has the amino acid sequence of SEQ ID NO: 8.

Alternatively or in addition, the present IL-2 variant term as IL-2_#8 may have: an amino acid sequence at least 85% identical to SEQ ID NO: 1; preferably, an amino acid sequence at least 90% identical to SEQ ID NO: 1; more preferably, an amino acid sequence at least 95% identical to SEQ ID NO: 1; even more preferably, the amino acid sequence of SEQ ID NO: 1; with the following amino acid substitutions: the amino acid substitution of phenylalanine (F) with valine (V) at the position corresponding to the position 42 in SEQ ID NO: 1, the amino acid substitution of tyrosine (Y) with serine(S) at the position corresponding to the position 45 in SEQ ID NO: 1, the amino acid substitution of leucine (L) with asparagine (N) at the position corresponding to the position 72 in SEQ ID NO: 1, and the amino acid substitution of cysteine (C) with glycine (G) at the position corresponding to the position 125 in SEQ ID NO: 1. In one working embodiment, the present IL-2 variant IL-2_#8 has the amino acid sequence of SEQ ID NO: 9.

Optionally, the present IL-2 variant term as IL-2_#9 may have: an amino acid sequence at least 85% identical to SEQ ID NO: 1; preferably, an amino acid sequence at least 90% identical to SEQ ID NO: 1; more preferably, an amino acid sequence at least 95% identical to SEQ ID NO: 1; even more preferably, the amino acid sequence of SEQ ID NO: 1; with the following amino acid substitutions: the amino acid substitution of phenylalanine (F) with histidine (H) at the position corresponding to the position 42 in SEQ ID NO: 1, the amino acid substitution of tyrosine (Y) with aspartic acid (D) at the position corresponding to the position 45 in SEQ ID NO: 1, the amino acid substitution of leucine (L) with isoleucine (I) at the position corresponding to the position 72 in SEQ ID NO: 1, and the amino acid substitution of cysteine (C) with glycine (G) at the position corresponding to the position 125 in SEQ ID NO: 1. In one working embodiment, the present IL-2 variant IL-2_#9 has the amino acid sequence of SEQ ID NO: 10.

In a further embodiment of the present disclosure, the present IL-2 variant term as IL-2_#12 may have: an amino acid sequence at least 85% identical to SEQ ID NO: 1; preferably, an amino acid sequence at least 90% identical to SEQ ID NO: 1; more preferably, an amino acid sequence at least 95% identical to SEQ ID NO: 1; even more preferably, the amino acid sequence of SEQ ID NO: 1; with the following amino acid substitutions: the amino acid substitution of phenylalanine (F) with cysteine (C) at the position corresponding to the position 42 in SEQ ID NO: 1, the amino acid substitution of tyrosine (Y) with asparagine (N) at the position corresponding to the position 45 in SEQ ID NO: 1, and the amino acid substitution of leucine (L) with valine (V) at the position corresponding to the position 72 in SEQ ID NO: 1. In one working embodiment, the present IL-2 variant IL-2_#12 has the amino acid sequence of SEQ ID NO: 13.

In yet some embodiments of the present disclosure, the present IL-2 variant term as IL-2_#25 may have: an amino acid sequence at least 85% identical to SEQ ID NO: 1; preferably, an amino acid sequence at least 90% identical to SEQ ID NO: 1; more preferably, an amino acid sequence at least 95% identical to SEQ ID NO: 1; even more preferably, the amino acid sequence of SEQ ID NO: 1; with the following amino acid substitutions: the amino acid substitution of alanine (A) with isoleucine (I) at the position corresponding to the position 50 in SEQ ID NO: 1. In one working embodiment, the present IL-2 variant IL-2_#25 has the amino acid sequence of SEQ ID NO: 21.

In other embodiments of the present disclosure, the present IL-2 variant term as IL-2_#28 may have: an amino acid sequence at least 85% identical to SEQ ID NO: 1; preferably, an amino acid sequence at least 90% identical to SEQ ID NO: 1; more preferably, an amino acid sequence at least 95% identical to SEQ ID NO: 1; even more preferably, the amino acid sequence of SEQ ID NO: 1; with the following amino acid substitutions: the amino acid substitution of phenylalanine (F) with alanine (A) at the position corresponding to the position 42 in SEQ ID NO: 1, the amino acid substitution of tyrosine (Y) with aspartic acid (D) at the position corresponding to the position 45 in SEQ ID NO: 1, and the amino acid substitution of leucine (L) with aspartic acid (D) at the position corresponding to the position 72 in SEQ ID NO: 1. In one working embodiment, the present IL-2 variant IL-2_#28 has the amino acid sequence of SEQ ID NO: 24.

Occasionally, the present IL-2 variant term as IL-2_#31 may have: an amino acid sequence at least 85% identical to SEQ ID NO: 1; preferably, an amino acid sequence at least 90% identical to SEQ ID NO: 1; more preferably, an amino acid sequence at least 95% identical to SEQ ID NO: 1; even more preferably, the amino acid sequence of SEQ ID NO: 1; with the following amino acid substitutions: the amino acid substitution of phenylalanine (F) with alanine (A) at the position corresponding to the position 42 in SEQ ID NO: 1, the amino acid substitution of tyrosine (Y) with methionine (M) at the position corresponding to the position 45 in SEQ ID NO: 1, and the amino acid substitution of leucine (L) with asparagine (N) at the position corresponding to the position 72 in SEQ ID NO: 1. In one working embodiment, the present IL-2 variant IL-2 #31 has the amino acid sequence of SEQ ID NO: 27.

In yet other embodiments of the present disclosure, the present IL-2 variant term as IL-2_#31_Y45R may have: an amino acid sequence at least 85% identical to SEQ ID NO: 1; preferably, an amino acid sequence at least 90% identical to SEQ ID NO: 1; more preferably, an amino acid sequence at least 95% identical to SEQ ID NO: 1; even more preferably, the amino acid sequence of SEQ ID NO: 1; with the following amino acid substitutions: the amino acid substitution of phenylalanine (F) with alanine (A) at the position corresponding to the position 42 in SEQ ID NO: 1, the amino acid substitution of tyrosine (Y) with arginine (R) at the position corresponding to the position 45 in SEQ ID NO: 1, and the amino acid substitution of leucine (L) with asparagine (N) at the position corresponding to the position 72 in SEQ ID NO: 1. In one working embodiment, the present IL-2 variant IL-2_#31_Y45R has the amino acid sequence of SEQ ID NO: 34.

The IL-2 variant of the present disclosure may be prepared by deletion, substitution, insertion, or modification using genetic or chemical methods well known in the art. Genetic methods may include site-specific mutagenesis of the encoding DNA sequence, polymerase chain reaction (PCR), gene synthesis, CRISPR/cas9 gene editing, and the like. The correct nucleotide changes may be verified for example by sequencing. In this regard, the nucleotide sequence of native human IL-2 is available from public database such as GenBank (GenBank deposited No.: S77834.1). The amino acid sequence of native human IL-2 is shown in SEQ ID NO: 1. Substitution or insertion may involve natural as well as non-natural amino acid residues. Amino acid modification includes well known methods of chemical modification such as the addition of glycosylation sites or carbohydrate attachments, and the like.

The present IL-2 variant may be obtained, for example, by solid-state peptide synthesis or recombinant production. For recombinant production, one or more polynucleotides encoding said IL-2 variant are isolated and inserted into one or more vectors for further cloning and/or expression in a host cell. Such polynucleotide may be readily isolated and sequenced using conventional procedures. Methods which are well known to those skilled in the art may be used to construct expression vectors containing the coding sequence of the present IL-2 variant along with appropriate transcriptional/translational control signals. These methods include in vitro recombinant DNA techniques, synthetic techniques, and in vivo recombination/genetic recombination. The expression vector may be part of a plasmid, virus, or may be a nucleic acid fragment. The expression vector includes an expression cassette into which the polynucleotide encoding the present IL-2 variant is cloned in operable association with a promoter and/or other transcription or translation control elements. A promoter may be operably associated with a nucleic acid encoding a polypeptide if the promoter is capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, may be operably associated with the polynucleotide to direct cell-specific transcription.

In certain embodiments the amino acid mutation present in the IL-2 variant may reduce the affinity of the present IL-2 variant to the α-subunit of the IL-2 receptor by at least 5-fold, such as by at least 10-fold, at least 25-fold, at least 30-fold, at least 50-fold, or even at least 100-fold. In one embodiment the amino acid mutation present in the IL-2 variant may abolish the affinity of the present IL-2 variant to the α-subunit of the IL-2 receptor. In the meantime, substantially similar binding to the intermediate-affinity receptor, i.e. preservation of the affinity of the present IL-2 variant to said receptor, is achieved when the IL-2 variant exhibits greater than about 70%, such as greater than about 80%, or greater than about 90%, of the affinity of a wild-type IL-2 to the intermediate-affinity IL-2 receptor.

In a specific embodiment, the present IL-2 variant may elicit one or more of the cellular responses such as proliferation in an activated T lymphocyte cell, differentiation in an activated T lymphocyte cell, cytotoxic T cell (CTL) activity, proliferation in an activated. B cell, differentiation in an activated B cell, proliferation in a NK cell, differentiation in a NK cell, cytokine secretion by an activated T cell or an NK cell, or NK/LAK antitumor cytotoxicity. In one embodiment the present IL-2 variant may reduce IL-2 signaling in T_{reg} cells, may reduce activation-induced cell death (AICD) in T cells, may have a reduced toxicity profile in vivo, or may have a prolonged serum half-life, as compared to wild-type IL-2.

The present IL-2 variant may be identified, screened for, or characterized for their physical/chemical properties and/or biological activities by various assays known in the art.

For example, the physical/chemical properties such as binding affinity assay may be measured. Specifically, the binding affinity of the present IL-2 variant for the IL-2 receptor may be easily determined by enzyme-linked immunosorbent assay (ELISA), by surface plasmon resonance (SPR) using a BIAcore instrument (GE Healthcare), or by using cell lines known to express one or the other form of the IL-2 receptor following the well-established procedures known in the art.

For biological activity assay, the activity of the present IL-2 variant may be indirectly measured by assaying the effects of immune activation that occur downstream of receptor binding, which may include, for example, the ability to induce proliferation of IL-2 receptor-bearing T and/or NK cells, the ability to induce IL-2 signaling in IL-2 receptor-bearing T and/or NK cells, the ability to generate IFN-γ by NK cells, a reduced ability to induce expression of IL-10 or TNF-α by peripheral blood mononuclear cells (PBMCs), a reduced ability to induce apoptosis in T cells, the ability to induce tumor regression and/or improve survival, and a reduced toxicity profile (e.g., reduced vasopermeability in vivo), via the methods known in the art.

Alternatively, the activity assay may be achieved by assaying the downstream signaling triggered by IL-2, which includes Janus kinase (JAK) and signal transducer and activator of transcription (STAT) signaling molecules. For example, phosphorylation of JAK1 and JAK3, as well as STAT-5 may be assessed as an indicator of activation of the IL-2 signaling, after the interaction of IL-2 with the IL-2 receptor β- and γ_{c}-subunits. Details of this method are disclosed in the Examples. T cells are treated with the present IL-2 variant, and the level of phosphorylated STAT5 is determined by flow cytometry.

### 2. The Pharmaceutical Composition

Also encompassed in the present disclosure is a pharmaceutical composition comprising the foregoing IL-2 variant for treating a cancer, and/or alleviating or ameliorating the symptoms associated with/caused by a cancer. In particular, the pharmaceutical composition comprises an effective amount of the IL-2 variant as described in any aspects or embodiments of the present disclosure; and optionally, a pharmaceutically acceptable carrier.

The IL-2 variant of this invention is present at a level of about 0.1% to 99% by weight, based on the total weight of the pharmaceutical composition. In some embodiments, the IL-2 variant of this invention is present at a level of at least 1% by weight, based on the total weight of the pharmaceutical composition. In certain embodiments, the IL-2 variant of this invention is present at a level of at least 5% by weight, based on the total weight of the pharmaceutical composition. In still other embodiments, the IL-2 variant of this invention is present at a level of at least 10% by weight, based on the total weight of the pharmaceutical composition. In still yet other embodiments, the IL-2 variant of this invention is present at a level of at least 25% by weight, based on the total weight of the pharmaceutical composition.

The present pharmaceutical composition is prepared following the pharmaceutical procedures known in the art, and may be formulated into solid, semi-solid, or liquid forms, such as tablets, capsules, powders, granules, ointments, solutions, suspensions, and injections, compatible with the intended routes of administration. These pharmaceutical preparations are a further object of the invention. One of skilled person in the art is familiar with the various dosage forms that are suitable for use in each route. It is to be noted that the most suitable route in any given case would depend on the nature or severity of the disease or condition being treated.

A pharmaceutically acceptable carrier is any carriers which are relatively non-toxic and innocuous to a patient at concentrations consistent with effective activity of the active ingredient so that any side effects ascribable to the carrier do not vitiate the beneficial effects of the active ingredient. Pharmaceutically acceptable carriers according to the invention are for example disintegrants, binders, lubricants, fillers, plasticizers, surfactants and wetting agents, film-forming agents and coating materials, and coloring agents for example pigments.

Disintegrants include, but are not limited to, croscarmellose sodium, crospovidone, alginic acid, carboxymethylcellulose calcium, carboxymethylcellulose sodium, microcrystalline cellulose, hydroxypropyl cellulose, low substituted hydroxypropyl cellulose, polacrillin potassium, cross-linked polyvinylpyrrolidone, sodium alginate, sodium starch glycollate, partially hydrolysed starch, sodium carboxymethyl starch, and starch.

Binders include, but are not limited to, hydroxypropyl cellulose, hydroxypropyl methylcellulose (HPMC), microcrystalline cellulose, acacia, alginic acid, carboxymethylcellulose, ethylcellulose, methylcellulose, hydroxaethylcellulose, ethylhydroxyethylcellulose, polyvinyl alcohol, polyacrylates, carboxymethylcellulose calcium, carboxymethylcellulose sodium, compressible sugar, ethylcellulose, gelatin, liquid glucose, methylcellulose, polyvinyl pyrrolidone, and pregelatinized starch.

Lubricants include, but are not limited to, calcium stearate, magnesium stearate, mineral oil, stearic acid, fumaric acid, sodium stearylfumarate, zinc stearate, and polyethyleneglycol.

Fillers include, but are not limited to, dibasic calcium phosphate, kaolin, lactose, mannitol, microcrystalline cellulose, silicated microcrystalline cellulose, dicalcium phosphate, tricalcium phosphate, magnesium trisilicate, mannitol, maltitol, sorbitol, xylitol, lactose for example the anhydrous form or the hydrate form such as the monohydrate form, dextrose, maltose, saccharose, glucose, fructose or maltodextrine, powdered cellulose, precipitated calcium carbonate, sodium carbonate, sodium phosphate, and starch.

Surfactants and wetting agents include, but are not limited to, heptadecaethylene oxycetanol, lecithins, sorbitol monooleate, polyoxyethylene sorbitol monooleate, polyoxyethylene stearate, polyoxyethylen sorbitan monolaurate, benzalkonium chloride, nonoxynol 10, oxtoxynol 9, polysorbates for example 20, 40, 60 or 80, sorbitan monopalmitate, sodium lauryl sulfate, sodium dodecylsulfate, sodium dioctylsulfosuccinate, glycerine monostearate, sorbitan monolaurate, polyethyleneglycol sorbitan monolaurate, polyethyleneglycol sorbitan monostearate, polyethyleneglycol sorbitan monooleate, copolymers of ethylenoxide and propylenoxide (PLURONIC®), and ethoxylated triglycerides.

Film-forming agents and coating materials include, but are not limited to, liquid glucose, hydroxyethyl cellulose, hydroxypropyl cellulose, HPMC, methylcellulose, ethylcellulose, cellulose acetate phthalate, shellac, polyvinylpyrrolidone, copolymers of vinylpyrrolidone and vinylacetate (KOLLIDON®), copolymers of dimethylaminomethacrylic acid and neutral methacrylic acid esters, polymers of methacrylic acid or methacrylic acid esters, copolymers of acrylic acid ethylester and methacrylic acid methyl ester, and copolymers of acrylic acid and acrylic acid methylester.

Plasticizers include, but are not limited to polyethylene glycol, diethyl phthalate, and glycerol.

Coloring agents include, but are not limited to pigments, inorganic pigments, FD&C Red No. 3, FD&C Red No. 20, FD&C Yellow No. 6, FD&C Blue No. 2, D&C Green No. 5, D&C Orange No. 5, D&C Red No. 8, caramel, ferric oxide red, ferric oxide yellow, and titanium dioxide.

Further commonly used pharmaceutical carriers may be used as appropriate to formulate the composition for its intended route of administration include, but is not limited to, acidifying agents (e.g., acetic acid, citric acid, fumaric acid, hydrochloric acid, or nitric acid); alkalizing agents (e.g., ammonia solution, ammonium carbonate, diethanolamine, mono-ethanolamine, potassium hydroxide, sodium borate, sodium carbonate, sodium hydroxide, triethanolamine, or trolamine); adsorbents (e.g., powdered cellulose, or activated charcoal); stabilizers and antioxidants (e.g., ascorbic acid, ascorbyl palmitate, butylated hydroxy-anisole, butylated hydroxytoluene, hypophosphorus acid, monothioglycerol, propyl gallate, sodium ascorbate, sodium bisulfite, sodium formaldehyde sulfoxylate, or sodium metabisulfite); other binding materials (e.g., block polymers, natural and synthetic rubber, polyacrylates, polyurethanes, silicones, polysiloxanes, or styrene-butadiene copolymers); buffering agents (e.g., potassium metaphosphate, dipotassium phosphate, sodium acetate, sodium citrate anhydrous, or sodium citrate hydrates); encapsulating agents (e.g., gelatin, starch, or cellulose derivates); flavorants, masking agents and odors (e.g., anise oil, cinnamon oil, cocoa, menthol, orange oil, peppermint oil, or vanillin); humectants (e.g., glycerol, propylene glycol, or sorbitol); sweeteners (e.g., aspartame, dextrose, glycerol, mannitol, propylene glycol, saccharin sodium, sorbitol, or sucrose); anti-adherents (e.g., magnesium stearate, or talc); direct compression excipients (e.g., dibasic calcium phosphate, lactose, or microcrystalline cellulose); tablet polishing agents (e.g., carnauba wax, or white wax).

The present pharmaceutical composition may further comprise other known pharmaceutically active agents (e.g., a chemotherapeutic agent, an immunotherapeutic agent, or a hormone therapeutic agent) to treat diseases and conditions caused by associated with a cancer.

Suitable chemotherapeutic agents for use in the present pharmaceutical composition may be one or more of actinomycin D, altretamine, aminoglutethimide, amsacrin, anastrozole, anthracycline, asparaginase, bendamustine, bexarotene, bleomycin, bortezomib, buserelin, busulfan, camptothecin, capecitabine, carboplatin, carmustine, chlorambucil, chlormethine, cisplatin, cladribine, clofarabine, clomifene, curcumin, cyclophosphamide, cytarabine, cytosinarabinoside, dacarbazine, dactinomycin, daunorubicin, dexamethasone, docetaxel, doxorubicin, epirubicin, estramustine, estrone, estradiol, estriol, etoposide, etoposid, exemestane, fludarabine, fluorouracil, formestane, foxuridine, gemcitabine, glucocorticoid, goserelin, hycamtin, hydroxy urea, idarubicin, ifosfamid, imatinib, indirubin, irinotecan, ixabepilone, letrozole, leuprorelin, lomustine, mechlorethamine, melphalan, mercaptopurine, methotrexate, miltefosin, mitomycine, mitoxantrone, nintedanib, nimustine, oxaliplatin, paclitaxel, pentostatin, plicamycin, prednisone, procarbazine, progesterone, raloxifene, streptozocin, tamoxifen, temozolomide, teniposide, testolactone, testosterone, thiotepa, thioguanine, topotecan, treosulfan, tretinoin, triptorelin, trofosfamide, vinblastine, vincristine, vindesine, or vinorelbine.

Said immunotherapeutic agent is selected from the group consisting of CCL3, CCL26, CXCL7; G-CSF, GM-CSF, IFN-α, IFN-β, IFN-γ, TNF-α; IL-4, IL-5, IL-7, IL-10, IL-12, IL-13, IL-15; apremilast, imiquimod, lenalidomide, pomalidomide, sipuleucel-T, thalidomide; anti-CTLA-4 antibody, anti-PD-1 antibody, anti-PD-L1 antibody; anti-CD2 antibody, anti-CD3 antibody, anti-CD4 antibody, anti-CD11a antibody, anti-CD20 antibody, anti-CD25 antibody, anti-CD52 antibody, anti-EGFR antibody, anti-HER2 antibody, anti-PCDP1 antibody, anti-SLAMF7 antibody, and anti-Trop-2 antibody. According to one preferred embodiment of the present disclosure, the additional immunotherapeutic agent used in the present pharmaceutical composition is IL-15.

Examples of the hormone therapeutic agent include, but are not limited to, androgen receptor agonists (e.g., androstenediol dipropionate, boldenone undecylenate, clostebol, or cloxotestosterone acetate); progonadotropins (e.g., bicalutamide, tamoxifen, clomifene, gonadorelin, or leuprorelin); androgen receptor antagonists (e.g., abiraterone acetate, canrenone, chlormadinone acetate, cyproterone acetate, or delmadinone acetate); steroidogenesis inhibitors (e.g., alfatradiol, dutasteride, epristeride, finasteride, flutamide, ketoconazole, or nilutamide); antigonadotropins (e.g., domperidone, metoclopramide, risperidone, haloperidol, chlorpromazine, sulpiride, testosterone, metoclopramide, or risperidone); estrogen receptor agonists (e.g., alfatradiol, testosterone, methyltestosterone, metandienone, or estradiol); estrogen receptor antagonists (e.g., acolbifene, anordrin, bazedoxifene, broparestrol, clomifene, or cyclofenil); aromatase inhibitors (e.g., aminoglutethimide, testolactone, fadrozole, formestane, anastrozole, exemestane, or letrozole); progesterone receptor agonists (e.g., allylestrenol, dienogest, norethisterone, progesterone, quingestrone, anagestone acetate, chlormadinone acetate, or chlormethenmadinone acetate); progesterone receptor antagonists (e.g., aglepristone, or mifepristone).

### 3. The Method for Treating Cancers

Another aspect of the present disclosure pertains to a method for treating a cancer in a subject. The method comprises administering to the subject an effective amount of the present IL-2 variant, or the pharmaceutical composition comprising the IL-2 variant.

The effective dose of the present IL-2 variant or the pharmaceutical composition administered to the subject may be ranging from about 0.01 μg/kg body weight to 1,000 mg/kg body weight of the subject, such as 0.01, 0.02, 0.03, 0.04, 0.05, 0.06, 0.07, 0.08, 0.09, 0.1, 0.2, 0.3 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1,000 μg/kg body weight (=1 mg/kg body weight), 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1,000 mg/kg body weight of the subject; preferably, about 0.1 to 1000 μg/kg body weight of the subject; more preferably, about 1 to 500 μg/kg body weight of the subject; even more preferably, about 10 to 200 μg/kg body weight of the subject. In some working examples, the effective dose of the present IL-2 variant or the pharmaceutical composition administered to the subject is 15, 50, or 150 μg/kg body weight of the subject. The dose can be administered in a single aliquot, or alternatively in more than one aliquot. The skilled artisan or clinical practitioner may adjust the dosage or regime in accordance with the physical condition of the patient or the severity of the diseases.

As would be appreciated, in the case when the subject afflicted with a cancer, the present method may be applied to the subject alone or in combination with additional therapies (e.g., a surgery, a radiotherapy, a chemotherapy, an immunotherapy, a hormone therapy, or a combination therapy thereof) that have some beneficial effects on the treatment of the cancer. Depending on the intended therapeutic purposes, the present method may be applied to the subject prior to, in conjunction with, or subsequent to the administration of the additional therapies. Among the foregoing additional therapies, the chemotherapy, the immunotherapy, the hormone therapy refer to administering to the subject an effective amount of a chemotherapeutic agent, an immunotherapeutic agent, or a hormone therapeutic agent, as described above; for the sake of brevity, the detail of those agents is omitted herein.

Said cancer may be any one of bladder cancer, biliary cancer, bone cancer, brain tumor, breast cancer, cervical cancer, colorectal cancer, esophageal cancer, epidermal carcinoma, gastric cancer, gastrointestinal stromal tumor (GIST), glioma, hematopoietic tumors of lymphoid lineage, hepatic cancer, Kaposi's sarcoma, leukemia, lung cancer, lymphoma, intestinal cancer, melanoma, myeloid leukemia, pancreatic cancer, prostate cancer, retinoblastoma, ovary cancer, renal cell carcinoma, spleen cancer, squamous cell carcinoma, thyroid cancer, or thyroid follicular cancer.

It should be noted that during the term of the present treatment, different therapies or therapeutics may be administered to the subject at different doses, time intervals, via different routes. The doses and time intervals may vary with factors as described above, and are dependent on the professional considerations of the practitioner; and the routes may be via oral, enteral, buccal, nasal, transdermal, transmucosal, intravenous, intraperitoneal, intraarterial, intracutaneous, subcutaneous, and intramuscular routes.

Basically, the subject treatable by the present method is a mammal; and preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Materials and Methods

### 1. Design and Cloning of the IL-2 Variants

For designing the IL-2 variants used herein, structures of both the receptor-bound active form and the inactive monomer form of IL-2 were first obtained from the online database: Protein Data Bank (PDB), with the PDB entry 2B5I or 2ERJ for the active form, and 1M4C or 3INK for the inactive form. The structures of both the active and the inactive forms of IL-2 were aligned by a commercial software (PyMOL), and the distances between the spatial locations of the specific atoms of the amino acids on helix A, C, and D in both the active and the inactive forms of IL-2 were calculated, thereby the atoms with the most and the least changes in the spatial locations being identified based on comparison of the structures of the active with the inactive forms of IL-2. The distances between the significantly shifted and the relatively not shifted atoms were calculated accordingly to create the signatures between the active and the inactive forms of IL-2. In the present study, the distances between the 50 atoms with the most changes in the spatial locations and the 60 atoms with the least changes in the spatial locations were calculated, resulting in 3000 (50×60) distances, in which the greatest of the calculated distances were selected to serve as the signatures of IL-2 from the inactive form becoming to the active form. On the other hand, the entire repertoire of the human IL-2 variants at F42, Y45, and L72 were 7,999 (20×20×20−1 (wild-type)) sequences, in which the structure of each of the IL-2 variants was predicted by an online software (AlphaFold Colab). With the knowledge of the structures of the active form of IL-2 and the IL-2 variants, the spatial distances between these two were calculated in accordance with the aforementioned calculative method that was performed between the active and the inactive forms of IL-2. IL-2 variants with the most atoms having <10% distance from the active form of IL-2 were selected, to serve as the present IL-2 variants (SEQ ID NOs: 1-29) for further studies; the sequences of these IL-2 variants were then subjected to artificial synthesis before cloning.

### 2. Expression and Verification of the IL-2 Variants

The constructs for prokaryotic expressing each of the above IL-2 variants (with hexahistidines (his6) tag; his6-IL-2 in pET) were transformed into the E. coli strain BL21 (DE3), and the IL-2 variants were expressed by induction from adding 0.1 mM IPTG into the lysogeny broth (LB)/ampicillin (50 μg/mL) culture when OD₆₀₀ reached 0.5-0.9, followed by cultivation for 16 hours at room temperature. The bacteria were harvested and lysed in 0.1 mL lysis buffer (20 mM HEPES, pH 7.4, 250 mM KCl, 25 μg/ml DNase, 25 μg/mL lysozyme, 10 mM PMSF, 10 mM β-ME), before the resulting mixture were subjected to centrifugation to collect the supernatants containing the expressed proteins. The soluble his6-IL-2 in the supernatants was separated by SDS-PAGE (10% polyacrylamide) gels, and was confirmed by western blotting using an anti-his6 antibody. The supernatants were then filtered and purified with a nickel-resin column (Qiagen, Valencia, CA) following the manufacturer's instructions. The amounts of the purified IL-2 variants were determined using an ELISA kit (human IL-2 Quantikine, R&D System).

The above IL-2 variants were also expressed in mammalian protein expression system (ExpiCHO™ expression system kit; Thermo). The above IL-2 variants in pcDNA3.4 were transfected into the ExpiCHO-S cells, and the reagent ExpiFectamine™ CHO enhancer was added into the culture on Day 1 of post-transfection. On Day 7, the ExpiCHO-S cells were removed by centrifugation, and the supernatant containing the proteins of the IL-2 variants was collected, whose concentration was determined by ELISA.

### 3. Cell Cultures

CTLL-2 cells (mouse cytotoxic T lymphocytes) and HH cells (human T lymphoblasts) were cultured in Roswell Park Memorial Institute (RPMI) 1640 medium supplemented with 10% fetal calf serum and 2 mM glutamine; and KPC-luc cells (mouse pancreatic ductal adenocarcinoma cells which were stably transfected with plasmid encoding both blasticidin-S deaminase and firefly luciferase) were cultured in the above medium with further supplement with 4 ng/ml of blasticidin. All the cells were incubated at 37° C. under a humidified atmosphere of 5% CO₂.

4. Measurement of pSTAT-5 Level in the IL-2 Variant-Stimulated Cells

To evaluate the activation of CTLL-2 or HH cells after being stimulated with the present IL-2 variants, the level of phosphorylation of STAT-5 (pSTAT-5) in the stimulated CTLL-2 and HH cells (2×10⁵ cells/well) was determined by flow cytometry. CTLL-2 and HH cells were incubated with wild-type IL-2 (proleukin), the indicated IL-2 variants, or the mutant IL-2 (i.e., IL-2v (AAG), in which AAG refers to F42A, Y45A, and L72G substitutions of IL-2; F. Hoffmann-La Roche Ltd)) at a single dose of 30 nM or a series of concentrations between 0-30 nM for 4 hours, depending on the purpose of the experiment. The treated CTLL-2 and HH cells were placed onto a 96-well microplate, in which each well was pre-seeded with KPC-luc cells (8×10³ cells/well), and then incubated for 30 minutes before being fixed with 2% formaldehyde (v/v) for 15 minutes at room temperature (RT). The fixed cells were permeabilized, rehydrated, incubated with an anti-phosphorylated STAT5 (pSTAT-5) antibody for 16 hours at 4° C., and then incubated with a FITC-conjugated anti-rabbit IgG for 60 minutes in the dark. The fluorescent signals were detected using a flow cytometer (BD FACS Calibur flow cytometer), and the data were analyzed using a software (CellQuest Pro and Cytexpert software; Beckman Coulter).

### 5. Animal Experiments

Female Balb/c mice (at 6 weeks of age) were maintained at 23±1° C. and at humidity of 45-70% with a 12 hour dark/light cycle. The mice were randomly divided into three groups (n=5 per group): the PBS control group, the human recombinant IL-2 (IL-2) control group, and the IL-2_#7 group. For the PBS group, the mice received intraperitoneal dosing according to the following schedule: a single daily dose for five consecutive days, followed by a two-day rest period; this cycle was repeated three times in total. For the IL-2 and the IL-2_#7 groups, the mice received intraperitoneal dosing according to the following schedule: (1) a single daily dose of 3 μg/mouse (about 0.15 mg/kg) for five consecutive days, followed by a two-day rest period; (2) a single daily dose of 10 μg/mouse (about 0.5 mg/kg) for five consecutive days, followed by a two-day rest period; and (3) a single daily dose of 30 μg/mouse (about 1.5 mg/kg) for five consecutive days, followed by a two-day rest period. After completion of the above dosing schedules, the animals were weighed and sacrificed, and their lungs, livers, and spleens were harvested for further analyses.

### 6. Flow Cytometry

The spleens of the mice were lysed to extract the cells therein. The cells were collected and fixed with 2% formaldehyde (v/v) for 15 minutes at RT, before being subjected to flow cytometry analysis. To identify particular cell populations in the spleens, the following antibody combinations were utilized: (1) for active CD8⁺ T cells: a FITC-conjugated anti-CD45 antibody (anti-CD45-FITC), an APC-conjugated anti-CD3 antibody (anti-CD3-APC), a PE-cy7-conjugated anti-CD8 antibody (anti-CD8-PE-cy7), and a PE-conjugated anti-Tbet antibody (anti-Tbet-PE); (2) for active NK cells: a PE-conjugated anti-CD45 antibody (anti-CD45-PE), an APC-conjugated anti-CD3 antibody (anti-CD3-APC), a FITC-conjugated anti-KN1.1 antibody (anti-KN1.1-FITC), and an AlexaFluor 700-conjugated anti-granzyme B (GrB) antibody (anti-GrB-AlexaFluor 700); and (3) for T_{reg} cells: an APC-cy7-conjugated anti-CD45 antibody (anti-CD45-APC-cy7), a FITC-conjugated anti-CD4 antibody (anti-CD4-FITC), an APC-conjugated anti-CD25 antibody (anti-CD25-APC), and a PE-conjugated anti-Foxp3 antibody (anti-Foxp3-PE). The fluorescent signals were detected using a flow cytometer (BD FACS Calibur flow cytometer), and the data were analyzed using a software (CellQuest Pro and Cytexpert software; Beckman Coulter).

### 7. Statistics

All the experiments were conducted in triplicate. Data were presented as mean±standard deviation (SD). Statistical significance was analyzed using the Student's t-test. For all statistical analyses, a two-tailed p-value <0.05 was considered significant. Calculations and diagrams were generated using the software GraphPad Prims 6.01.

### Example 1 Production and Characterization of the IL-2 Variants

### 1.1 Production of the IL-2 Variants

The aim of this example was to identify novel IL-2 variants that possess a strong binding ability toward the intermediate-affinity IL-2R rather than the high-affinity IL-2R, so as to trim the unwanted immunosuppression effects associated with the α-subunit of the IL-2R. For the foregoing purpose, several IL-2 variants were designed and produced by the procedures described in the section of “Materials and Methods.” The nucleic acid sequence encoding each IL-2 variants was confirmed by sequencing (data not shown), and the amino acid sequence of each IL-2 variants was provided in the sequence listing. These IL-2 variants were further characterized for their biological activities as described below.

### 1.2 Biological Activities of the IL-2 Variants

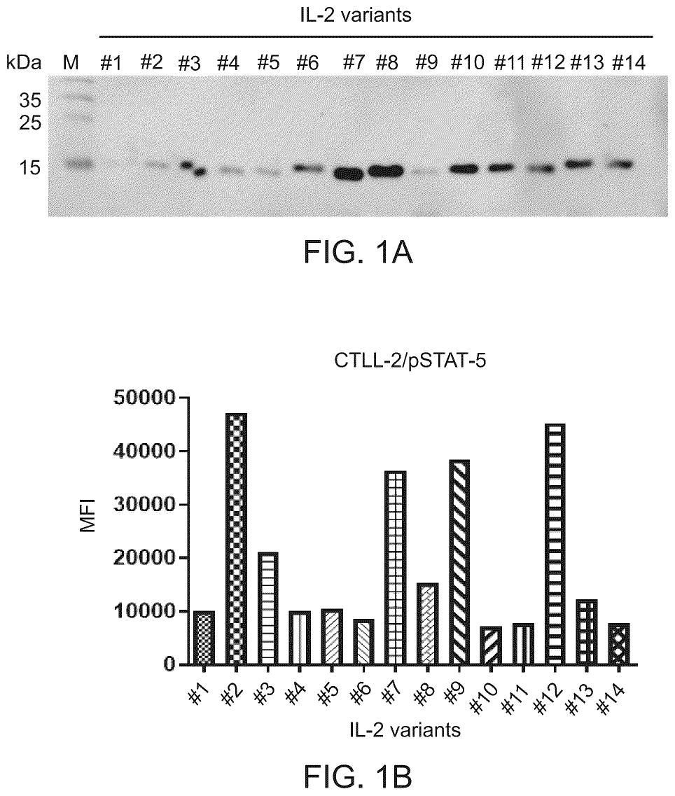

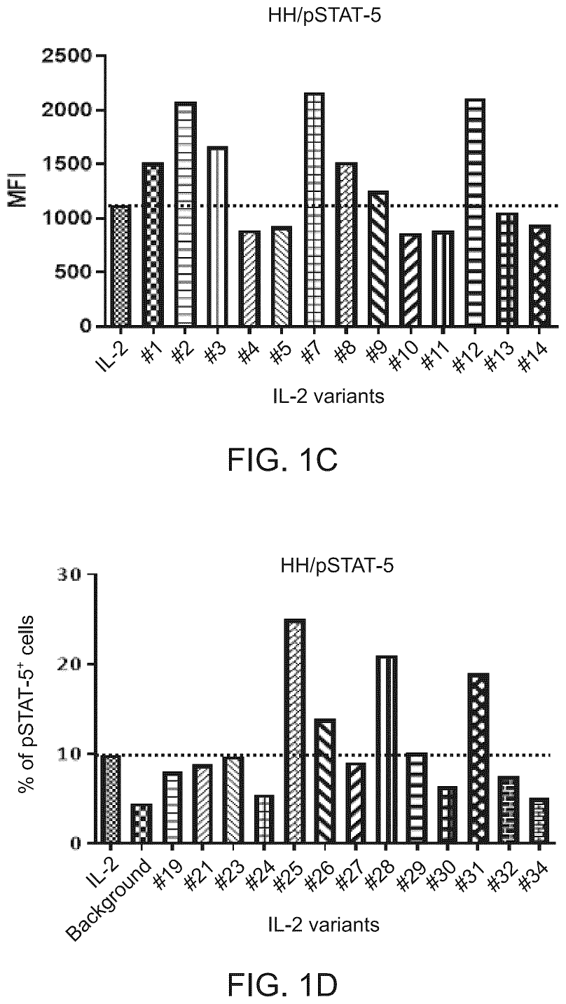

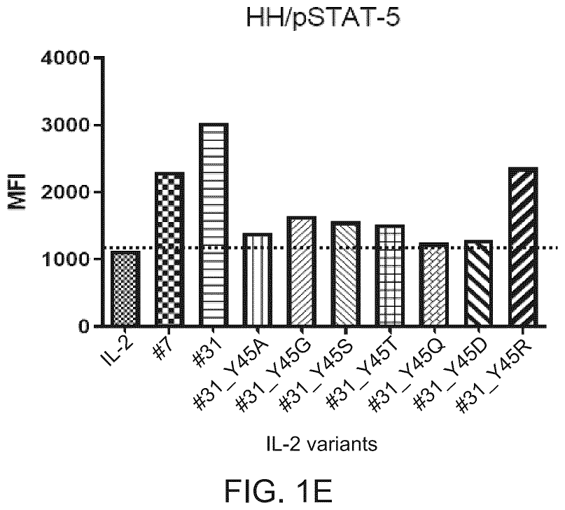

First, the expression of the IL-2 variants (designated as IL-2_#1 to IL-2_#14) of Example 1.1 were confirmed by western blot analysis, and the results were illustrated in FIG. 1A, in which each IL-2 variants (about 15 kDa) was successfully expressed.

Then, the biological activity of each IL-2 variants was evaluated via its ability to activate CTLL-2 or HH cells (in the presence of KPC-luc cells), and the degree of the activation of the CTLL-2 or HH cells was presented as the level of the phosphorylation of STAT-5 induced in the cells. As shown in FIGS. 1B-1C, the IL-2 variants could activate CTLL-2 cells (FIG. 1B) or HH cells (FIG. 1C); among them, IL-2_#2, IL-2_#3, IL-2_#7, IL-2_#8, IL-2_#9, and IL-2_#12 consistently exhibited higher activation power for both CTLL-2 cells and HH cells (i.e., exhibiting higher levels of pSTAT-5) as compared to those of IL-2_#1, IL-2_#4-6, IL-2_#10-11, and IL-2_#13-14. Similarly, the IL-2 variants (designated as IL-2_#19 to IL-2_#34) also possessed the ability to activate HH cells to varying degrees, and IL-2_#25, IL-2_#28, and IL-2_#31 exhibited higher activation power for HH cells (i.e., higher percentage of pSTAT-5-positive cells; FIG. 1D) as compared to that of the rest of IL-2 variants.

Further, IL-2_#31 (with F42A, Y45M, and L72N substitutions of IL-2) was chosen as the exemplary IL-2 variant for the investigation of the effect of amino acid substitution at specific site of IL-2 on the activation of HH cells. Note that in the following variants associated with IL-2_#31 (hereafter, “the IL-2_#31 variants”), the tyrosine (Y) at the position 45 of IL-2 was substituted with alanine (A) (Y45A), aspartic acid (D) (Y45D), glycine (G) (Y45G), glutamine (Q) (Y45Q), arginine (R) (Y45R), serine(S) (Y45S), or threonine (T) (Y45T), whereas F42A and L72N as in IL-2_#31 remained in the above IL-2_#31 variants (SEQ ID NOs: 30-36). The results were as shown in FIG. 1E, in which only the variant Y45R of IL-2_#31 (i.e., IL-2_#31_Y45R) exhibited improved activity in HH cells, as compared to that of the other variants. These results indicated that amino acid variations at specific sites in a specified IL-2 variant may drastically affect the activation of IL-2 in HH cells. These IL-2 variants (i.e., having cell activation ability) were then subjected to further analysis.

### 1.3 Binding Preference of the IL-2 Variants

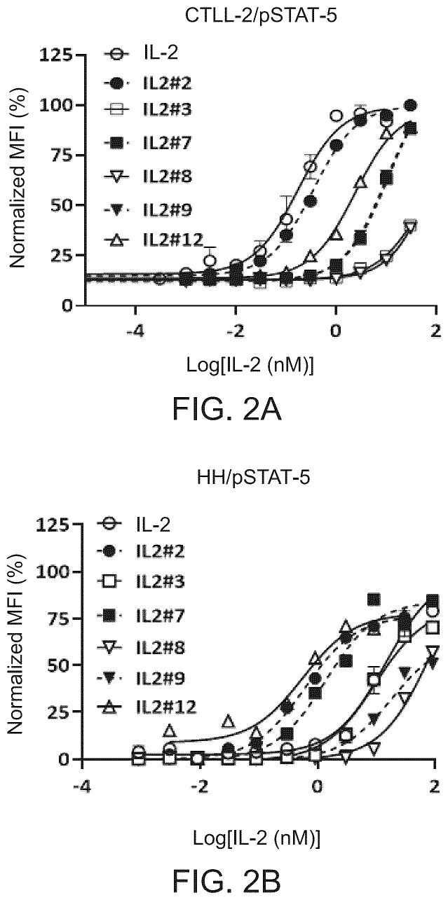

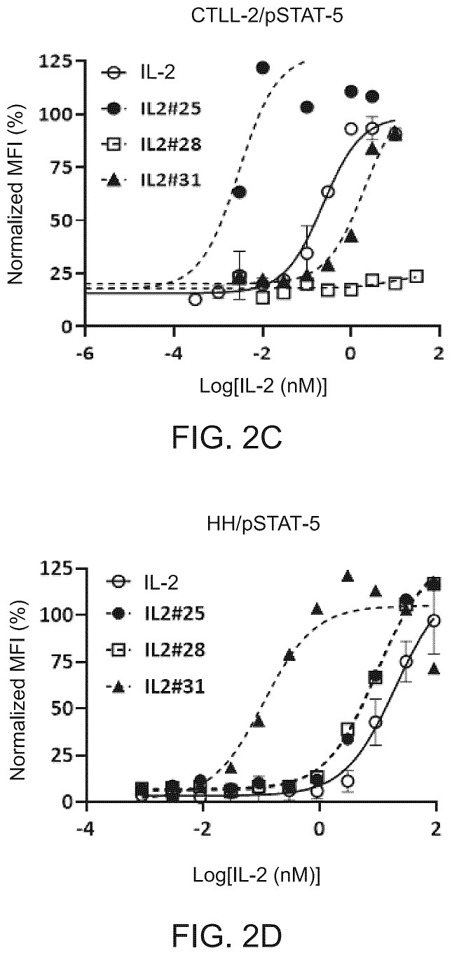

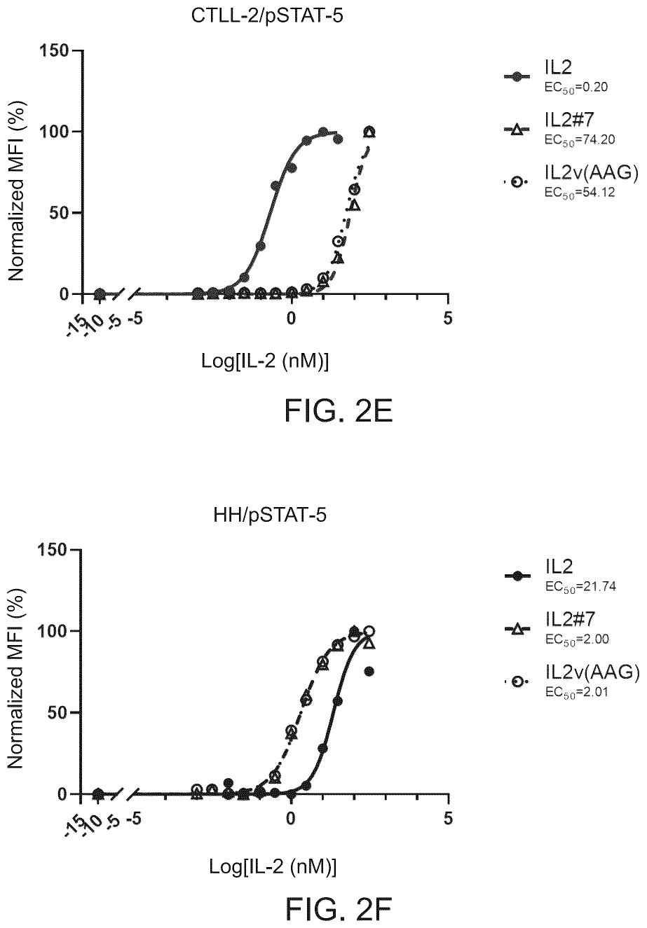

The purpose of this Example was to clarify the binding preference of the IL-2 variants of Example 1.1 toward the high-affinity IL-2R or the intermediate-affinity IL-2R. To this end, the level of pSTAT-5 in the stimulated CTLL-2 cells and HH cells (after treating a series concentration of the indicated IL-2 variants) was used as an indicator of the activation of the cells, in which the CTLL-2 cells express the high-affinity IL-2R (containing the α/β/γ_{c}-subunits of the IL-2R), whereas the HH cells express the intermediate-affinity IL-2R (containing the β/γ_{c}-subunits of the IL-2R). The results for activation by IL-2_#2, IL-2_#3, IL-2_#7, IL-2_#8, IL-2_#9, and IL-2_#12 in the CTLL-2 and HH cells were provided in FIGS. 2A-2B, respectively. The results were expressed as normalized MFI (%), which was relative and normalized to IL-2 (as designated as 100%); the EC₅₀ of the above IL-2 variants for activating the CTLL-2 cells (designated as EC₅₀ (CTLL-2)) and the HH cells (designated as EC₅₀ (HH)) were calculated accordingly, and are summarized in Tables 1-2. The binding preference of the IL-2 variants toward different forms of IL-2R was expressed as EC₅₀ (HH/CTLL-2), and summarized in Table 3.

TABLE 1
The EC₅₀ of IL-2_#2 to IL-2_#12 for CTLL-2 cells

| Name | IL-2 | IL-2_#2 | IL-2_#3 | IL-2_#7 | IL-2_#8 | IL-2_#9 | IL-2_#12 |
|---|---|---|---|---|---|---|---|
| EC₅₀ (nM) | 0.1700 | 0.3416 | 44.11 | 11.50 | 120.2 | 10.01 | 2.323 |

TABLE 2
The EC₅₀ of IL-2_#2 to IL-2_#12 for HH cells

| Name | IL-2 | IL-2_#2 | IL-2_#3 | IL-2_#7 | IL-2_#8 | IL-2_#9 | IL-2_#12 |
|---|---|---|---|---|---|---|---|
| EC₅₀ (nM) | 14.24 | 0.6122 | 9.600 | 1.356 | 90.18 | 17.91 | 0.5563 |

TABLE 3
The relative EC₅₀ of IL-2_#2 to IL-2_#12 for CTLL-2 and HH cells

| Name | IL-2 | IL-2_#2 | IL-2_#3 | IL-2_#7 | IL-2_#8 | IL-2_#9 | IL-2_#12 |
|---|---|---|---|---|---|---|---|
| EC₅₀ (HH) | 14.24 | 0.6122 | 9.600 | 1.356 | 90.18 | 17.91 | 0.5563 |
| EC₅₀ | 0.1700 | 0.3416 | 44.11 | 11.50 | 120.2 | 10.01 | 2.323 |
| (CTLL-2) | | | | | | | |
| EC₅₀ | 83.76 | 1.79 | 0.22 | 0.12 | 0.75 | 1.79 | 0.24 |
| (HH/CTLL-2) | | | | | | | |

According to Table 3, the EC₅₀ (HH/CTLL-2) value of IL-2 was 83.76, suggesting IL-2 preferred the high-affinity IL-2R over the intermediate-affinity IL-2R. By contrast, IL-2_#2 to IL-2_#12 exhibited relatively smaller EC₅₀ (HH/CTLL-2) as compared to that of IL-2, indicating that these IL-2 variants prefer the intermediate-affinity IL-2R than the high-affinity IL-2R.

FIGS. 2C-2D illustrates the respective activation powers of IL-2_#25, IL-2_#28, and IL-2_#31 to CTLL-2 and HH cells; while the binding preferences results are summarized in Tables 4-6.

TABLE 4
The EC₅₀ of IL-2_#25 to IL-2_#31 for CTLL-2 cells

| Name | IL-2 | IL-2_#25 | IL-2_#28 | IL-2_#31 |
|---|---|---|---|---|
| EC₅₀ (nM) | 0.2309 | 0.002908 | 9.562 | 2.018 |

TABLE 5
The EC₅₀ of IL-2_#25 to IL-2_#31 for HH cells

| Name | IL-2 | IL-2_#25 | IL-2_#28 | IL-2_#31 |
|---|---|---|---|---|
| EC₅₀ (nM) | 19.66 | 9.933 | 8.949 | 0.1099 |

TABLE 6
The relative EC₅₀ of IL-2_#25 to
IL-2_#31 for CTLL-2 and HH cells

| Name | IL-2 | IL-2_#25 | IL-2_#28 | IL-2_#31 |
|---|---|---|---|---|
| EC₅₀ (HH) | 19.66 | 9.933 | 8.949 | 0.1099 |
| EC₅₀ (CTLL-2) | 0.2309 | 0.002908 | 9.562 | 2.018 |
| EC₅₀ (HH/CTLL-2) | 85.48 | 3424.14 | 0.94 | 0.05 |

According to Table 6, IL-2 preferred the high-affinity IL-2R over the intermediate-affinity IL-2R, by contrast, both IL-2_#28 and IL-2_#31 preferred the intermediate-affinity IL-2R over the high-affinity IL-2R, as the EC₅₀ (HH/CTLL-2) for IL-2_#28 and IL-2_#31 were 0.94 and 0.05, respectively. Nonetheless, the EC₅₀ (HH/CTLL-2) value of IL-2_#25 was 3424.14, much higher than that of IL-2, suggesting that IL-2_#25 was more prone to bind with the high-affinity IL-2R.

The binding preference towards CTLL-2 and HH cells by IL-2_#7 was compared with that by the commercially available mutant IL-2 (IL-2v (AAG); F. Hoffmann-La Roche Ltd). The results are shown in FIGS. 2E-2F and summarized in Table 7. According to the results, IL-2_#7 (EC₅₀ (HH/CTLL-2)=0.027) exhibited a superior binding preference towards HH cells compared to wild-type IL-2 (EC₅₀ (HH/CTLL-2)=10.817) or IL-2v (AAG) (EC₅₀ (HH/CTLL-2)=0.037). This suggests that IL-2_#7 performs better in binding to the intermediate-affinity IL-2R than to the high-affinity IL-2R, as compared to the commercial IL-2v (AAG).

TABLE 7
The EC₅₀ and relative EC₅₀ of IL-2_#7 and IL-2v(AAG)

| Name | IL-2 | IL-2_#7 | IL-2v(AAG) |
|---|---|---|---|
| EC₅₀ (HH) | 21.74 | 2.00 | 2.01 |
| EC₅₀ (CTLL-2) | 0.20 | 74.20 | 54.12 |
| EC₅₀ (HH/CTLL-2) | 108.7 | 0.027 | 0.037 |

### Example 2 In Vivo Toxicity and Immune Modulation of the IL-2 Variants

### 2.1 the Toxicity of the IL-2 Variants

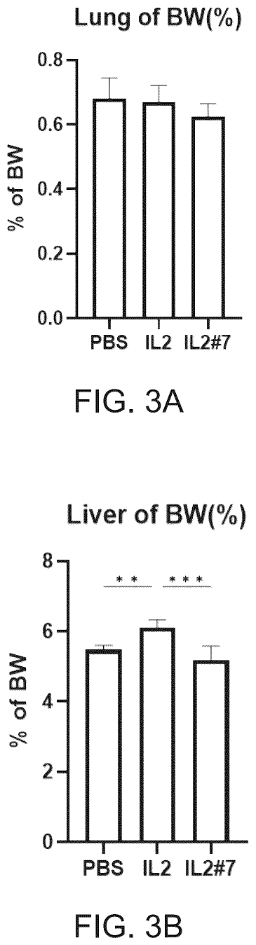

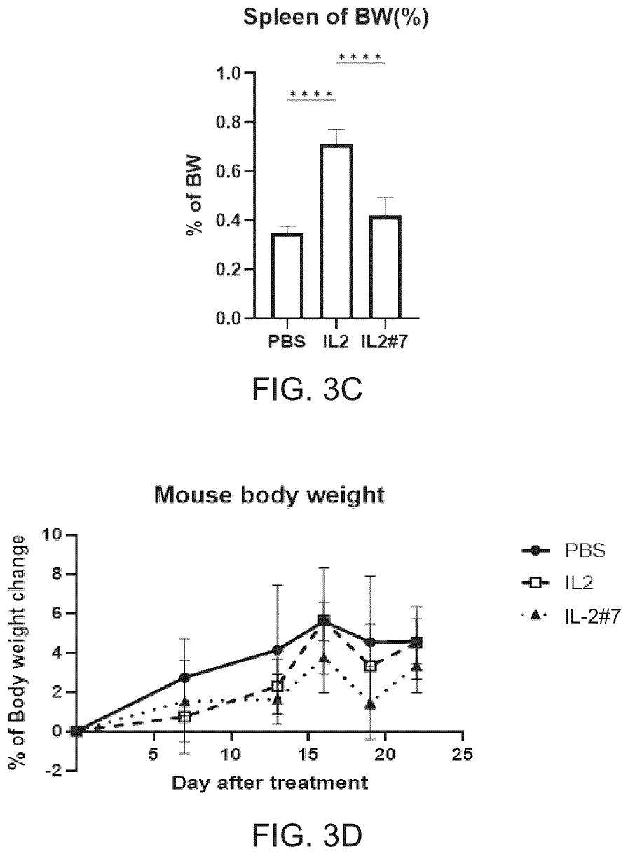

In the present example, the in vivo toxicity and immune modulation of the IL-2 variants were investigated. The mice were administered with the indicated IL-2 variants in accordance with the treatment schedules described in the “Materials and Methods.” After completion of the treatment schedules, the mice were sacrificed. Considering the detrimental impact of IL-2 on endothelial cells leading to organ edema, such as in the lung, liver, and spleen, resulting in weight changes, these organs were weighed and normalized against individual body weights (FIGS. 3A-3C). Furthermore, the changes in body weights over the course of the experiment were presented in FIG. 3D. The relative liver and spleen weights of mice treated with wild-type IL-2 significantly increased compared to those treated with IL-2_#7, indicating that IL-2_#7 has lower toxic effects in the treated mice. In this study, the relative lung weights remained relatively the same among the three treatment groups. Further, the body weights of the mice in these treatment groups maintained consistent body weights throughout the study. Taken together, these data indicated that IL-2_#7 exhibited a better safety profile compared to wild-type IL-2.

### 2.2 the Immune Modulation of the IL-2 Variants

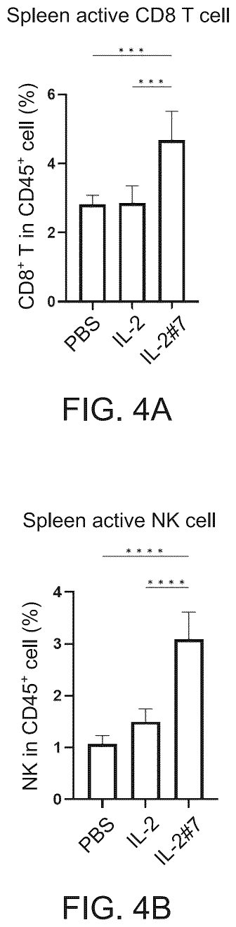

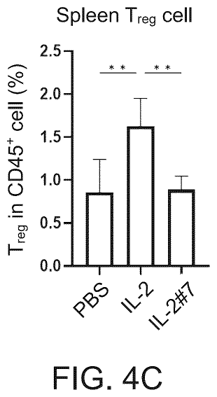

Next, the immune modulation ability of the IL-2 variants was evaluated in this study, which could be reflected by changes in the quantity of immune cells in peripheral lymphoid tissues (e.g., spleen). To this end, the quantity of the active CD8⁺ T cells, the active NK cells, and the T_{reg} cells were measured by flow cytometry. Based on FIGS. 4A-4B, active CD8⁺ T cells (marked by CD45⁺, CD3⁺, CD8⁺, and T-bet+) and active NK cells (marked by CD45⁺, GrB+, NK1.1⁺, and CD3⁻) increased significantly in the IL-2_#7 treatment group compared to those in the PBS and IL-2 treatment groups. In contrast, the IL-2_#7 treatment group did not show an increase in T_{reg} cell population (marked by CD45⁺, Foxp3⁺, CD4⁺, and CD25⁺). Collectively, these data demonstrated that IL-2_#7 might promote a beneficial immune response by stimulating the production of active CD8⁺ T and NK cells, without provoking adverse immune response by activating the production of T_{reg} cells. As such, IL-2_#7 has the potential to enhance an individual's immune system, making it a promising candidate for development as an anti-cancer therapeutic.

Taken together, the present invention provides several IL-2 variants, and many of them exhibit binding preference toward the intermediate-affinity IL-2R, which in turn trigger the biological activities associated therefrom. Thus, these IL-2 variants could eliminate the unwanted property of immunosuppression while preserving desired biological activities, thus are of great potential for the development of medicaments for treating IL-2 associated diseases.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. An isolated interleukin-2 (IL-2) variant having an amino acid sequence at least 85% identical to SEQ ID NO: 1, wherein the isolated IL-2 variant comprises an amino acid substitution at a position corresponding to the positions 42, 45, 50, 72, or 125 in SEQ ID NO: 1, in which:
the amino acid substitution at the position corresponding to the position 42 in SEQ ID NO: 1 is a substitution of phenylalanine (F) with alanine (A), cysteine (C), glutamic acid (E), histidine (H), valine (V), or tryptophan (W);
the amino acid substitution at the position corresponding to the position 45 in SEQ ID NO: 1 is a substitution of tyrosine (Y) with alanine (A), aspartic acid (D), glycine (G), methionine (M), asparagine (N), glutamine (Q), arginine (R), serine(S), or threonine (T);
the amino acid substitution at the position corresponding to the position 50 in SEQ ID NO: 1 is a substitution of alanine (A) with isoleucine (I);
the amino acid substitution at the position corresponding to the position 72 in SEQ ID NO: 1 is a substitution of leucine (L) with aspartic acid (D), isoleucine (I), lysine (K), asparagine (N), or valine (V); and
the amino acid substitution at the position corresponding to the position 125 in SEQ ID NO: 1 is a substitution of cysteine (C) with glycine (G).

2. The isolated IL-2 variant of claim 1, wherein the isolated IL-2 variant comprises the amino acid substitution of phenylalanine (F) with tryptophan (W) at the position corresponding to the position 42 in SEQ ID NO: 1, and the amino acid substitution of leucine (L) with lysine (K) at the position corresponding to the position 72 in SEQ ID NO: 1.

3. The isolated IL-2 variant of claim 1, wherein the isolated IL-2 variant comprises the amino acid substitution of phenylalanine (F) with valine (V) at the position corresponding to the position 42 in SEQ ID NO: 1, the amino acid substitution of tyrosine (Y) with alanine (A) at the position corresponding to the position 45 in SEQ ID NO: 1, the amino acid substitution of leucine (L) with asparagine (N) at the position corresponding to the position 72 in SEQ ID NO: 1, and the amino acid substitution of cysteine (C) with glycine (G) at the position corresponding to the position 125 in SEQ ID NO: 1.

4. The isolated IL-2 variant of claim 1, wherein the isolated IL-2 variant comprises the amino acid substitution of phenylalanine (F) with glutamic acid (E) at the position corresponding to the position 42 in SEQ ID NO: 1, and the amino acid substitution of tyrosine (Y) with glycine (G) at the position corresponding to the position 45 in SEQ ID NO: 1.

5. The isolated IL-2 variant of claim 1, wherein the isolated IL-2 variant comprises the amino acid substitution of phenylalanine (F) with valine (V) at the position corresponding to the position 42 in SEQ ID NO: 1, the amino acid substitution of tyrosine (Y) with serine(S) at the position corresponding to the position 45 in SEQ ID NO: 1, the amino acid substitution of leucine (L) with asparagine (N) at the position corresponding to the position 72 in SEQ ID NO: 1, and the amino acid substitution of cysteine (C) with glycine (G) at the position corresponding to the position 125 in SEQ ID NO: 1.

6. The isolated IL-2 variant of claim 1, wherein the isolated IL-2 variant comprises the amino acid substitution of phenylalanine (F) with histidine (H) at the position corresponding to the position 42 in SEQ ID NO: 1, the amino acid substitution of tyrosine (Y) with aspartic acid (D) at the position corresponding to the position 45 in SEQ ID NO: 1, the amino acid substitution of leucine (L) with isoleucine (I) at the position corresponding to the position 72 in SEQ ID NO: 1, and the amino acid substitution of cysteine (C) with glycine (G) at the position corresponding to the position 125 in SEQ ID NO: 1.

7. The isolated IL-2 variant of claim 1, wherein the isolated IL-2 variant comprises the amino acid substitution of phenylalanine (F) with cysteine (C) at the position corresponding to the position 42 in SEQ ID NO: 1, the amino acid substitution of tyrosine (Y) with asparagine (N) at the position corresponding to the position 45 in SEQ ID NO: 1, and the amino acid substitution of leucine (L) with valine (V) at the position corresponding to the position 72 in SEQ ID NO: 1.

8. The isolated IL-2 variant of claim 1, wherein the isolated IL-2 variant comprises the amino acid substitution of alanine (A) with isoleucine (I) at the position corresponding to the position 50 in SEQ ID NO: 1.

9. The isolated IL-2 variant of claim 1, wherein the isolated IL-2 variant comprises the amino acid substitution of phenylalanine (F) with alanine (A) at the position corresponding to the position 42 in SEQ ID NO: 1, the amino acid substitution of tyrosine (Y) with aspartic acid (D) at the position corresponding to the position 45 in SEQ ID NO: 1, and the amino acid substitution of leucine (L) with aspartic acid (D) at the position corresponding to the position 72 in SEQ ID NO: 1.

10. The isolated IL-2 variant of claim 1, wherein the isolated IL-2 variant comprises the amino acid substitution of phenylalanine (F) with alanine (A) at the position corresponding to the position 42 in SEQ ID NO: 1, the amino acid substitution of tyrosine (Y) with methionine (M) at the position corresponding to the position 45 in SEQ ID NO: 1, and the amino acid substitution of leucine (L) with asparagine (N) at the position corresponding to the position 72 in SEQ ID NO: 1.

11. The isolated IL-2 variant of claim 1, wherein the isolated IL-2 variant comprises the amino acid substitution of phenylalanine (F) with alanine (A) at the position corresponding to the position 42 in SEQ ID NO: 1, the amino acid substitution of tyrosine (Y) with arginine (R) at the position corresponding to the position 45 in SEQ ID NO: 1, and the amino acid substitution of leucine (L) with asparagine (N) at the position corresponding to the position 72 in SEQ ID NO: 1.

12. The isolated IL-2 variant of claim 1, wherein the isolated IL-2 variant comprises the amino acid sequence of SEQ ID NO: 1.

13. A pharmaceutical composition comprising the isolated IL-2 variant of claim 1, and a pharmaceutically acceptable carrier.

14. A method for treating a cancer in a subject, comprising administering to the subject an effective amount of the pharmaceutical composition of claim 13.

15. The method of claim 14, wherein the pharmaceutical composition comprising the isolated IL-2 variant is administered to the subject in the amount of 1-500 μg/kg body weight.

16. The method of claim 14, wherein the cancer is any one of bladder cancer, biliary cancer, bone cancer, brain tumor, breast cancer, cervical cancer, colorectal cancer, esophageal cancer, epidermal carcinoma, gastric cancer, gastrointestinal stromal tumor (GIST), glioma, hematopoietic tumors of lymphoid lineage, hepatic cancer, Kaposi's sarcoma, leukemia, lung cancer, lymphoma, intestinal cancer, melanoma, myeloid leukemia, pancreatic cancer, prostate cancer, retinoblastoma, ovary cancer, renal cell carcinoma, spleen cancer, squamous cell carcinoma, thyroid cancer, or thyroid follicular cancer.

17. The method of claim 14, further comprising subjecting the subject to a surgery, a radiotherapy, a chemotherapy, an immunotherapy, a hormone therapy, or a combination therapy thereof, prior to, concurrently with, or subsequent to the administration of the pharmaceutical composition of claim 13.

18. The method of claim 17, wherein the immunotherapy comprises administering to the subject an effective amount of C-C motif chemokine ligand 3 (CCL3), C-C motif chemokine ligand 26 (CCL26), C-X-C motif chemokine ligand 7 (CXCL7); granulocyte colony-stimulating factor (G-CSF), granulocyte macrophage colony-stimulating factor (GM-CSF), interferon-α (IFN-α), interferon-β (IFN-β), interferon-γ (IFN-γ), tumor necrosis factor-α (TNF-α); interleukin-4 (IL-4), interleukin-5 (IL-5), interleukin-7 (IL-7), interleukin-10 (IL-10), interleukin-12 (IL-12), interleukin-13 (IL-13), interleukin-15 (IL-15); apremilast, imiquimod, lenalidomide, pomalidomide, sipuleucel-T, thalidomide; anti-CTLA-4 antibody, anti-PD-1 antibody, anti-PD-L1 antibody; anti-CD2 antibody, anti-CD3 antibody, anti-CD4 antibody, anti-CD11a antibody, anti-CD20 antibody, anti-CD25 antibody, anti-CD52 antibody, anti-EGFR antibody, anti-HER2 antibody, anti-PCDP1 antibody, anti-SLAMF7 antibody, or anti-Trop-2 antibody.

19. The method of claim 18, wherein the immunotherapy comprises administering to the subject an effective amount of IL-15.

20. The method of claim 14, wherein the subject is a human.
